(19) United States
(12) Patent Application Publication
(10) Pub. No.: US 2004/0248134 A1
(43) Pub. Date: Dec. 9, 2004

(54) Pre-existing nucleic acids covalently attached to a metal surface of a metal cluster, intermediates thereof and methods of using same

(76) Inventors: Joseph Sperling, Jerusalem (IL); Ohad Medalia, Moshav Yarkona (IL)

Correspondence Address: Anthony Castorina, G E Ehrlich, Suite 207, 2001 Jefferson Davis Highway, Arlington, VA, 22202, US

(21) Appl. No.: 10/492,537

(22) PCT Filed: Oct. 29, 2002
(86) PCT No.: PCT/IL02/00869

(30) Foreign Application Priority Data
Nov. 1, 2001 (IL) 146270

(51) Int. Cl. C12Q 1/68; C12M 1/34; B05D 3/00

(52) U.S. Cl. 435/6; 435/287.2; 427/2.11

(57) ABSTRACT

A method of covalently attaching a pre-existing nucleic acid to a metal surface or a metal cluster is disclosed. The method comprises chemically modifying the pre-existing nucleic acid, so as to obtain a functionallized pre-existing nucleic acid having a side chain terminating with a functional group, which side chain is covalently linked to a purine or pyrimidine base of the pre-existing nucleic acid, and covalently attaching the functionallized pre-existing nucleic acid to a metal surface or a metal cluster, via the functional groups. A method of preparing a thin and flat gold film is further disclosed.

### FIELD AND BACKGROUND OF THE INVENTION

The present invention relates to pre-existing nucleic acids covalently attached to a metal surface or a metal cluster and, more particularly, to a method of chemically modifying (chemically functionallizing) pre-existing, non-functionallized, nucleic acids and covalently attaching the chemically modified (functionallized) nucleic acids to a solid metal surface or to a metal cluster. The present invention further relates to a method of preparing a solid support that is highly suitable for the attachment of nucleic acids thereto and to uses of the products and methods of the present invention

The fast growing interest in gaining information about the structure and function of genes has promoted the development of high throughput methodologies for DNA expression analysis on DNA microarrays, also known as DNA chips (Cheung et al., 1999). Presently, several alternatives exist for the analysis of genetic information in a microarray format. The most common microarray platform exploits a solid substrate to which pieces of DNA are attached and is commonly referred to as a DNA chip. The presently used solid supports for DNA arrays typically include glass and various plastic materials. Functionallized versions of these supports (e.g., oligoehteleneglycol-glass, or charged (aminated) nylon) are commonly used to tether DNA or oligonucleotides to the surface through specific locations (Southern et al., 1999).

Alternative surfaces that can serve as advantageous supports for DNA microarrays are gold surfaces. The gold supports are highly advantageous since they are chemically inert to bio-macromolecules but, at the same time, accessible to chemisorption via the formation of stable, covalent gold-thiol bonds (Finklea, 1996; Sabatani and Rubinstein, 1987; Ulman, 1996).

Gold surfaces are typically prepared by evaporation of gold in high vacuum on various substrates such as silicon and mica. Gold films that are prepared at optimal conditions are textured films with preferential orientation and wide atomically flat terraces (Golan et al., 1992). Though these terraces are atomically flat, substantial height differences between terraces and deep grooves are frequently detected. This high roughness of the surfaces exerts a substantial limitation to this technique, especially in cases where the array is subjected to measurements such as atomic force microscopy, as is further detailed hereinbelow.

Template stripped gold (TSG) substrates (Hegner et al., 1993; Samori et al., 1999) are presently known as suitable gold surfaces, although their preparation is far more complicated. However, roughening (Knarr et al., 1998) or contamination of the gold surface with mica flakes (Thomson et al., 1999) may occur during the detaching process employed by this technique which may interfere with the process of sample adsorption thereon.

Hence, the presently known techniques for the preparation of gold surfaces as suitable substrates for DNA or other bio-macromolecule microarrays are limited, since they provide roughened and/or contaminated surfaces. There is thus a widely recognized need for, and it would be highly advantageous to have, gold surfaces devoid of the above limitations.

In recent years, atomic force microscopy and, in particular, tapping mode atomic force microscopy (TMAFM) has become a common tool for structural and physical studies of biological macromolecules, mainly because it provides the ability to perform experiments with samples in buffer solutions (Drake at al., 1989). The structure of proteins and nucleic acids can thus be studied in their physiological environment that allows native intermolecular complexes to be formed. AFM imaging requires the immobilization of the molecules on a support since the AFM tip that probes the sample's surface exerts on it substantial forces. Hence, for obtaining meaningful AFM imaging, it is essential that the interaction between the sample analyzed and the supporting surface will be stronger than that between the sample and the AFM imaging tip.

One of the presently common supports for proteins and DNA investigated by AFM is mica (Engel et al., 1999; Guthold et al., 1999). The macromolecules physisorb to the mica, mainly via electrostatics and van-der-Waals interactions. However, many bio-macromolecules do not spontaneously physisorb to, or detach from, mica surfaces under buffer conditions required for their biological activity. On the other hand, hydrophilic molecules, such as DNA, may bind so strongly to the hydrophilic mica surface and, as a result, are forced to undergo conformational changes due to binding at multiple sites. This limitation has been partially solved by covalent binding of bio-macromolecules through a specific location, which does not interfere with their biological activity (Delamarche et al., 1996; Thomson et al., 1999). Such a solution does not overcome the strong adherence of the nucleic acids to mica surfaces, and therefore this limitation can be better overcome using gold surfaces, which are highly hydrophobic, as supports for AFM imaging of DNA.

DNA interacts weakly with gold surfaces (Hegner et al., 1993) and therefore cannot be attached directly to gold surfaces and cannot be visualized in solution by TMAFM. Nevertheless, force spectroscopic measurements of base pair-unbinding force in DNA deposited on gold proved the existence of the DNA on the surface (Rief et al., 1999), which was attributed to the relatively high DNA concentration used and the drying steps that were performed. These conditions may induce topological constraints of the DNA, which may cause potential imaging problems. Furthermore, strong binding of the DNA to the surface, through non-specific interactions, may prevent further interactions between the DNA and other macromolecules, which is the main object of binding the DNA to a microarray. Therefore, it is recommended to anchor the DNA to the gold surface through its termini, which may further allow the imaging by TMAFM of single DNA molecules in their native state. However, due to lack of appropriate functional groups, the immobilization of nucleic acids onto gold surfaces is not as straight forward as the immobilization of proteins.

Since it is well known in the art that thiol groups interact strongly, via covalent bonds, with gold, several works were conducted in the field of introducing one or more thiol groups into DNA molecules and characterizing the interaction of the thiolated molecules with gold surfaces.

Thus, short oligonucleotides were anchored onto gold surfaces by preparing terminally thiolated oligonucleotides using solid phase DNA synthesizing techniques (Kelley et al., 1998). Immobilization of longer DNA fragments, prepared by PCR from thiolated oligonucleotide primers, was also achieved, though at low efficiency (Hegner et al., 1993).

Intensive work has been done in characterizing the binding of thiolated single-stranded DNA (ssDNA) (Steel et al., 2000) and double-stranded DNA (ddDNA) (Yang et al., 1998) onto gold surfaces. These studies teach that the orientation of the ssDNA on the gold surface is highly dependent on the length of the strand. These studies further teach that some of the amine groups of the nucleo-bases can weakly interact with gold surfaces.

In one example, the group of Steel (Steel et al., 2000) prepared 5′-thiolated 718 bp DNA by PCR amplification using 5′-thiolated oligodeoxynucleotides as primers. The thiolated synthetic DNA covalently interacted with the gold surface solely via its 5′-thiol group, while unmodified dsDNA could not be detected on the gold surface after washing steps, and therefore did not bind to the gold surface. Contrary to the dsDNA, ssDNA interacted with the gold surface either as a thiolated DNA or as unmodified DNA. Nevertheless, the interaction of the unmodified ssDNA was characterized as weak and reversible.

The possibility of hybridization and detection of DNA hybrids in an array-like technology by using chemically modified gold surfaces has been also described (Brockman et al., 1999). The detection of the DNA hybrids was performed using surface plasmon resonance spectroscopy (SPS) (Jordan et al., 1997) and atomic force microscopy (AFM) (He et al., 2000). The modification of the gold surfaces in these studies was conducted by covering the gold with self assembled monolayers that contained either primary amines (Jordan et al., 1997) or carboxylate groups (He et al., 2000). The obtained functional gold surfaces were then covalently cross-linked with oligodeoxynucleotides, which were thereafter hybridized with oligodeoxynucleotides conjugated to gold clusters for AFM measurements (He et al., 2000) or with biotin (Jordan et al., 1997). These studies teach a possibility to use arrays that are probed by SPS without the need for dyes, as well as a method of quantificating the hybridization product by AFM. Nevertheless, these studies do not teach DNA probes that use native DNA, and are further disadvantageous since the surface preparation in these studies includes complicated procedures, resulting in an activated surface which may bind non-specifically a variety of macromolecules, unless complicated blocking procedures are used.

The studies described hereinabove further demonstrate the advantage in using nascent, unmodified gold films for hybridization detection assays. The use of nascent gold films provides for the reversible absorption of mercapto-alkyl derivatives of ssDNA while dsDNA does not adsorb to the surface (Steel et al., 2000). In assays in which the sample DNA is double-stranded and it becomes single-stranded only upon denaturation (e.g., by heating), ssDNA does not bind to such gold surfaces and therefore the background in such systems is expected to be reduced significantly during analysis.

Other methods of thiolating nucleic acids are described in the art. Ghosh et al., (1990) attached a thiol group to the 5′ phosphate of oligonucleotides, in order to conjugate a desired sequence of nucleic acids to an enzyme and thus enable the molecule's detection by allowing signal amplification.

Hanna et al. used 5-[(4-azidophenacyl)thio]-UTP, 5-APAS-UTP (Hanna et al., 1989) and 5-APAS-CTP (Hanna et al., 1993) to study RNA-protein and RNA-nucleic acid interaction via photochemical cross-linking. These modified nucleotides contain azido groups, which are used to activate such cross-linking and can be introduced into RNA molecules by an in vitro transcription reaction. The precursors of these compounds were 5-thio-UTP and 5-thio-CTP, respectively. The same authors have further reported the synthesis of 5-thiodeoxyuridine, which can be incorporated in a specific location along DNA via solid phase DNA synthesis.

Goodwin and Glick (1993) have synthesized 5-alkylthiol-dUTP in order to study secondary structures of DNA and RNA through site specific formation of disulfide cross-linking. The DNA that contained these thiolated nucleotides was prepared by solid phase synthesizers but, as reported by the authors, could not be used for enzyme-catalyzed transcription or replication.

The synthesis and incorporation of 5-(2-mercaptoethyl)-UTP into RNA by enzyme-catalyzed transcription in vitro, was recently reported by Vaish et al., 2000. Although this work provides a detailed description of a thiolated compound that can be incorporated via in vitro transcription reaction, the synthesis described therein is rather complicated and includes attachment of the triphosphate group to the thiolated base and protection of the thiol group as the disulfide until used.

Medalia et al. (1999) and WO 01/20017 teach a strategy for introducing thiol groups into nucleic acids and their subsequent coupling to metal clusters. This strategy is based on the incorporation of nucleoside triphosphates carrying a terminally thiolated arm on their heterocyclic ring (the purine or pyrimidine base) into nucleic acids by a template directed enzymatic synthesis. Nevertheless, this work clearly fails to teach chemical (as opposed to enzymatic) thiolation of native, pre-existing, nucleic acids.

The coupling of nucleic acids to metal clusters, in particular gold clusters, is further described in the art. Such coupling is typically used for visualizing biological macromolecules. The presently most popular method employs colloidal gold non-covalently attached to specific antibodies, protein A, avidin, streptavidin or other macromolecular probes that high affinity bind a respective counterpart (i.e., members of binding pairs). For example, attempts were made to visualize spliceosomes by dark-field scanning transmission electron microscopy (STEM) after tagging with biotinylated oligonucleotides complementary to the pre-mRNA which had been conjugated to streptavidin-colloidal gold complex (Sibbald et al., 1993).

The use of probes with covalently conjugated gold compounds provides a number of advantages over colloidal gold. These include better stability, size uniformity, and complete absence of aggregation, all of which result in better sensitivity and resolution. A number of gold clusters containing a core of eleven gold atoms surrounded by a hydrophilic organic shell of aryl-phosphines has been described (Safer et al., 1986). The diameter of the undecagold cluster is 0.82 nm. It can thus be visualized by high-resolution STEM, but not readily by conventional TEM, unless the signal is highlighted by silver enhancement (Burry et al., 1992). Visualization by conventional EM can be improved by using larger, 1.4 nm, gold clusters (Hainfeld and Furuya, 1992). The structure of this reagent, now commercially available from Nanoprobe (Stony Brook NY) under the name “NANOGOLD”, has not yet been reported. However, it has been used successfully to label proteins (Boisset et al., 1992; Hainfeld and Furuya, 1992) as well as DNA oligonucleotides (Alivisatos et al., 1996). Nevertheless, gold clusters that are chemically attached directly to pre-existing nucleic acids have not been disclosed yet.

Hence, the prior art teaches several synthetic routes to incorporate thiol groups in nucleic acids. Nevertheless, all the presently known procedures to produce thiolated DNA or RNA use pre-thiolated nucleobases that are synthetically, either by solid phase synthesis or by enzymatic synthesis, incorporated into the macromolecules. The prior art clearly fails to teach methods of chemically thiolating and thereby chemically functionallizing, without any synthetic steps of elongation, pre-existing nucleic acids and, in particular, native nucleic acids.

The prior art teaches methods of binding modified nucleic acids onto gold surfaces or to gold clusters. These teachings include binding oligodeoxynucleotides to various types of gold surfaces. The prior art further teaches hybridization of the bound oligodeoxynucleotides with labeled nucleic acids or proteins. Nevertheless, the prior art fails to teach methods of modifying native, pre-existing DNA molecules so as to enable their binding to gold clusters or gold surfaces, as well as direct hybridization assays of nucleic acids attached to nascent gold supports.

There is thus a widely recognized need for, and it would be highly advantageous to have, a method of modifying a pre-existing, native nucleic acid and binding the modified nucleic acid to a solid support, particularly a gold support, and/or to a metal cluster. It would be further highly advantageous to have a method of preparing a gold support devoid the limitations of the presently known techniques.

### SUMMARY OF THE INVENTION

According to one aspect of the present invention there is provided a method of covalently attaching a pre-existing nucleic acid to a metal surface, the method comprising chemically modifying the pre-existing nucleic acid, so as to obtain a functionallized pre-existing nucleic acid having one or more functional group(s) at a termini of one or more side chain(s) that are covalently linked to one or more purine or pyrimidine base(s) of the pre-existing nucleic acid and attaching the functionallized pre-existing nucleic acid to a metal surface, so as to covalently attach the pre-existing nucleic acid to the metal surface via the one or more functional group(s).

According to another aspect of the present invention there is provided a pre-existing nucleic acid covalently attached to a metal surface by the method described hereinabove.

According to yet another aspect of the present invention there is provided a method of covalently attaching a pre-existing nucleic acid to a metal cluster, the method comprising chemically modifying the pre-existing nucleic acid, so as to obtain a functionallized pre-existing nucleic acid having one or more functional group(s) at a termini of one or more side chain(s) that are covalently linked to one or more purine or pyrimidine base(s) of the pre-existing nucleic acid and attaching the functionallized pre-existing nucleic acid to a metal cluster, so as to covalently attach the pre-existing nucleic acid to the metal cluster via the one or more functional group(s).

According to still another aspect of the present invention there is provided a pre-existing nucleic acid covalently attached to a metal cluster by the method described hereinabove.

According to an additional aspect of the present invention there is provided a method of preparing a nucleic acid chip presenting a pre-existing nucleic acid covalently attached thereto, the method comprising chemically modifying the pre-existing nucleic acid, so as to obtain a functionallized pre-existing nucleic acid having one or more functional group(s) at a termini of one or more side chain(s), which side chain(s) being covalently linked to one or more purine or pyrimidine base(s) of the pre-existing nucleic acid and attaching the functionallized pre-existing nucleic acid to a chip, so as to covalently attach the pre-existing nucleic acid to the chip via the one or more functional group(s).

According to yet an additional aspect of the present invention there is provided a nucleic acid chip presenting a pre-existing nucleic acid covalently attached thereto, prepared by the method described hereinabove.

According to still an additional aspect of the present invention there is provided a method of screening a nucleic acid chip presenting a pre-existing nucleic acid covalently attached thereto and interacted with a macromolecule, the method comprising obtaining a nucleic acid chip presenting a pre-existing nucleic acid covalently attached thereto, interacting the pre-existing nucleic acid with a macromolecule, so as to obtain the nucleic acid chip presenting the pre-existing nucleic acid covalently attached thereto and interacted with the macromolecule and screening the nucleic acid chip presenting the pre-existing nucleic acid covalently attached thereto and interacted with the macromolecule, for bound macromolecule.

According to further features in preferred embodiments of the invention described below, the nucleic acid chip comprises a metal surface.

According to still further features in the described preferred embodiments the step of obtaining the nucleic acid chip comprises chemically modifying the pre-existing nucleic acid, so as to obtain a functionallized pre-existing nucleic acid having one or more functional group(s) at a termini of one or more side chain(s), which side chain(s) being covalently linked to one or more purine or pyrimidine base(s) of the pre-existing nucleic acid and attaching the functionallized pre-existing nucleic acid to a metal surface, so as to covalently attach the pre-existing nucleic acid to the metal surface via the one or more functional group(s).

According to still further features in the described preferred embodiments the screening is selected from the group consisting of AFM imaging, fluorescence measuring, autoradiographing, transmission electron microscopy (TEM) scanning, dark-field scanning transmission electron microscopy (STEM), electron spectroscopic imaging (ESI), scanning tunneling microscopy (STM) and surface plasmon resonance spectroscopy (SPS). According to still further features in the described preferred embodiments the macromolecule is dye-labeled and the screening comprises fluorescence measurement.

According to still further features in the described preferred embodiments the macromolecule is labeled by a radioactive agent and the screening comprises autoradiographing.

According to still further features in the described preferred embodiments the macromolecule is labeled by a metal cluster and the screening is selected from the group consisting of transmission electron microscopy (TEM) scanning, surface plasmon resonance spectroscopy (SPS), dark-field scanning transmission electron microscopy (STEM) and AFM imaging.

According to a further aspect of the present invention there is provided a method of imaging a pre-existing nucleic acid, the method comprising chemically modifying the pre-existing nucleic acid, so as to obtain a functionallized pre-existing nucleic acid having one or more functional group(s) at a termini of one or more side chain(s), which side chain(s) being covalently linked to one or more purine or pyrimidine base(s) of the pre-existing nucleic acid, covalently attaching the functionallized pre-existing nucleic acid to a metal surface, so as to obtain the pre-existing nucleic acid covalently attached to the metal surface via the one or more functional group(s) and imaging the pre-existing nucleic acid covalently attached to the metal surface via the one or more functional group(s).

According to further features in preferred embodiments of the invention described below, the imaging is via atomic force microscopy.

According to still further features in the described preferred embodiments the imaging is via a screening method selected from the group consisting of transmission electron microscopy (TEM) scanning, dark-field scanning transmission electron microscopy (STEM), electron spectroscopic imaging (ESI), surface plasmon resonance spectroscopy (SPS) and scanning tunneling microscopy (STM). According to further features in preferred embodiments of the invention described below, the metal surface is a thin flat gold film prepared by evaporating a gold wire onto a chip under reduced pressure, while gradually heating the gold wire.

According to yet a further aspect of the present invention there is provided a method of preparing a thin flat gold film, the method comprising evaporating a gold wire onto a chip under reduced pressure, while gradually heating the gold wire.

According to further features in preferred embodiments of the invention described below, the chip is a mica chip.

According to still further features in the described preferred embodiments the gold film has a mean roughness that ranges between 0.1 nm and 1 nm.

According to still further features in the described preferred embodiments the reduced pressure ranges between 10⁻⁵ torr and 10⁻⁶ torr.

According to still further features in the described preferred embodiments the method further comprising washing the gold film with an organic solvent and drying the gold film.

According to still further features in the described preferred embodiments the pre-existing nucleic acid is RNA.

According to still further features in the described preferred embodiments the pre-existing nucleic acid is DNA.

According to still further features in the described preferred embodiments the pre-existing nucleic acid includes one or more base analog(s).

According to still further features in the described preferred embodiments the pre-existing nucleic acid is treated prior to the step of chemically modifying, so as to restrict chemical modifications to predetermined purine or pyrimidine bases in the pre-existing nucleic acid.

According to still further features in the described preferred embodiments the pre-existing nucleic acid is a double stranded DNA and is treated by a sequence specific endonuclease prior to the step of chemically modifying, so as to restrict chemical modifications to predetermined purine or pyrimidine bases in a 3′ or 5′ single stranded overhang generated by endonucleolysis.

According to still further features in the described preferred embodiments the pre-existing nucleic acid is a double stranded nucleic acid and is treated by a 3′ or 5′ specific exonuclease prior to the step of chemically modifying so as to restrict chemical modifications to predetermined purine or pyrimidine bases in a 5′ or 3′ single stranded overhang generated by the 3′ or 5′ specific exonucleolysis, respectively.

According to still further features in the described preferred embodiments the pre-existing nucleic acid is a single stranded nucleic acid and is treated by one or more complementary protecting polynucleotide(s) prior to the step of chemically modifying so as to restrict chemical modifications to predetermined purine or pyrimidine bases in one or more region(s) not protected by the one or more complementary protecting polynucleotide(s), following hybridization with the one or more complementary protecting polynucleotide(s).

According to still further features in the described preferred embodiments the chemically modifying comprises thiolating the pre-existing nucleic acid.

According to still further features in the described preferred embodiments the functional group is a thiol group.

According to still further features in the described preferred embodiments the step of chemically modifying comprises thiolating the is pre-existing nucleic acid, so as to obtain a thiolated pre-existing nucleic acid having a thiol functional group at the termini of the side chain being covalently linked to the one or more purine or pyrimidine base(s) of the nucleic acid.

According to still further features in the described preferred embodiments the side chain is saturated or unsaturated and has 2-20 carbon atoms.

According to still further features in the described preferred embodiments the saturated or unsaturated side chain has 2-10 carbon atoms.

According to still further features in the described preferred embodiments the saturated or unsaturated side chain is interrupted one or more heteroatom(s) selected from the group consisting of O, S and N and/or is substituted by one or more chemical group(s) selected from the group consisting of ═O, ═NH and an alkyl group having 1-3 carbon atoms.

According to still further features in the described preferred embodiments the surface is selected from the group consisting of a metal plate, a metal film and a metal coat.

According to still further features in the described preferred embodiments the metal is selected from the group consisting of Ag, Au, Hg, Pt, Mo and W.

According to still further features in the described preferred embodiments the metal is gold.

According to still further features in the described preferred embodiments the metal film is a thin gold film having a thickness ranging between 1 nm and 20 nm.

According to still further features in the described preferred embodiments the metal cluster is a gold cluster.

According to still further features in the described preferred embodiments the gold cluster is selected from the group consisting of a maleimido derivative of a gold cluster and colloidal gold of pre-determined size.

According to still further features in the described preferred embodiments the pre-existing nucleic acid is interacted with a macromolecule.

According to still further features in the described preferred embodiments the macromolecule is labeled.

According to still further features in the described preferred embodiments the macromolecule is of a biological source. According to still further features in the described preferred embodiments the macromolecule is a nucleic acid.

According to still further features in the described preferred embodiments the macromolecule is a protein.

According to still a further aspect of the present invention there is provided a method of thiolating a pre-existing nucleic acid, the method comprising covalently attaching a side chain terminating with a thiol group to one or more unpaired purine or pyrimidine base(s) of the pre-existing nucleic acid.

According to further features in preferred embodiments of the invention described below, the unpaired purine base is selected from the group consisting of an adenine base and a guanine base.

According to still further features in the described preferred embodiments the unpaired pyrimidine base is selected from the group consisting of a thymine base, a cytosine base and an uracil base.

According to still further features in the described preferred embodiments the pre-existing nucleic acid is treated prior to the covalently attaching, so as to obtain the one or more unpaired purine or pyrimidine base(s) of the pre-existing nucleic acid.

According to still further features in the described preferred embodiments the pre-existing nucleic acid is a double stranded DNA and is treated by a sequence specific endonuclease prior to the step of covalently attaching, so as to obtain the one or more unpaired purine or pyrimidine base(s) of the pre-existing nucleic acid in a 3′ or 5′ single stranded overhang generated by endonucleolysis.

According to still further features in the described preferred embodiments the pre-existing nucleic acid is a double stranded nucleic acid and is treated by a 3′ or 5′ specific exonuclease prior to the step of covalently attaching, so as to obtain the one or more unpaired purine or pyrimidine base(s) of the pre-existing nucleic acid in a 5′ or 3′ single stranded overhang generated by the 3′ or 5′ specific exonucleolysis, respectively.

According to still further features in the described preferred embodiments the pre-existing nucleic acid is a single stranded nucleic acid and is treated by one or more complementary protecting polynucleotide(s) prior to the step of covalently attaching, so as to obtain the one or more unpaired purine or pyrimidine base(s) of the pre-existing nucleic acid in one or more region(s) not protected by the one or more complementary protecting polynucleotide(s), following hybridization with the one or more complementary protecting polynucleotide(s).

The present invention successfully addresses the shortcomings of the presently known configurations by providing a novel method of covalently attaching a pre-existing nucleic acid to a metal surface or a metal cluster, which can be further interacted with a macromolecule. The method of the present invention can be used to prepare a nucleic acid chip presenting such a pre-existing nucleic acid and for imaging pre-existing nucleic acids. The present invention further provides a method of thiolating a pre-existing nucleic acid and a method of preparing a thin gold film devoid of the limitations of the presently known configurations.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-c are AFM images of 500 nm×500 nm areas of thin flat gold films prepared according to the method of the present invention, which have a gold surface thickness of 1 nm and RMS roughness of 0.2 nm, with black to white spans of 5 nm (FIG. 1a), a gold surface thickness of 6 nm and a RMS roughness of 0.5 nm, with black to white spans of 10 nm (FIG. 1b) and a thickness of 10 nm and a RMS roughness of 0.8 nm, with black to white spans of 12 nm (FIG. 1c);

FIG. 2 is a scheme describing the synthetic route to obtain pre-existing DNA fragments that are covalently attached to a gold surface, according to a preferred embodiment of the present invention;

FIGS. 3a-b are TMAFM images in solutions of gold films having a thickness that ranges between 4 nm and 6 nm, which were treated with unmodified DNA fragments (FIG. 3a) and with thiolated DNA fragments prepared by the method of the present invention (FIG. 3b);

FIGS. 4a-c are TMAFM images showing full-length tracings of DNA restriction fragments of 900 bp (FIGS. 4a and 4b) and 2450 bp (FIG. 4c) that were covalently attached to gold surfaces according to the method of the present invention;

FIG. 5 is a close-up view (an area of 300 nm×300 nm) of an AFM image showing DNA fragments that were covalently attached to a gold surface through their termini (marked by arrow heads), according to a preferred 15 embodiment of the present invention; and

FIGS. 6a-c are AFM images (770 nm×770 nm areas) of TSG surfaces, treated with a complex of SspC and an unmodified DNA (FIG. 6a), treated with a terminally thiolated DNA and then with SspC (FIG. 6b) and treated with a pre-formed SspC-thiolated DNA complex (FIG. 6c).

### DESCRIPTION OF THE PREFERRED EMBODIMENTS

The present invention is of a method of covalently attaching a pre-existing nucleic acid to a metal surface which can be used to prepare nucleic acid microarrays, such as nucleic acid chips, having a metal, e.g., gold, surface, and presenting pre-existing nucleic acids. Specifically, the present invention can be used to provide metal surfaces that present pre-existing nucleic acid covalently attached thereto, per se and also following interaction with other macromolecules. Hence, the present invention can be used for investigating pre-existing nucleic acids or complexes thereof with other macromolecules by various analytical methods and is particularly useful for AFM imaging. The present invention is also of a method of covalently binding a metal cluster to pre-existing nucleic acids and further of a method of preparing thin gold films, which can be used as surfaces for attaching nucleic acids.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Nucleic acid microarrays and, in particular, nucleic acid chips that present nucleic acids attached to solid surfaces, are one of the most powerful tools currently available for genome analysis and therefore play a significant role in the analysis of genome sequences, gene expression and other genetic parameters.

However, the presently known nucleic acids chips typically use oligonucleotides that are either synthesized in situ on the solid support or pre-synthesized and thereafter deposited on the support. Thus, the presently known nucleic acid ships are typically limited to synthetically prepared nucleic acids.

In sharp distinction, the present invention provides a method of covalently attaching pre-existing nucleic acids to a solid surface. This novel use of pre-existing nucleic acids in the field of microarrays is highly advantageous mainly since it enables the use of native (naturally occurring) nucleic acids and thus enables better understanding of the genetic mechanism as well as an ability to explore macromolecular assemblies containing native nucleic acids.

Thus, according to one aspect of the present invention, there is provided a method of covalently attaching a pre-existing nucleic acid to a metal surface. The method according to this aspect of the present invention is effected by chemically modifying a pre-existing nucleic acid, so as to obtain a functionallized pre-existing nucleic acid that has one or more functional group(s) at a termini of one or more side chain(s), which side chain being covalently linked to one or more purine or pyrimidine base(s) of the pre-existing nucleic acid, and attaching the functionallized pre-existing nucleic acid to a metal surface, so as to covalently attach the pre-existing nucleic acid to the metal surface via the functional group(s).

As used herein, the phrase “pre-existing nucleic acid” includes native or synthetic nucleic acids that are devoid of a functional group which can be used to covalently attach such pre-existing nucleic acids to a metal surface or cluster. Thus, a pre-existing nucleic acid can be RNA or DNA and can include native nucleobases as well as analog nucleobases, also known as base analogs.

The pre-existing nucleic acids are chemically modified, according to the method of the present invention, so as to obtain functionallized pre-existing nucleic acids.

As used herein the phrases “chemically modifying” and “chemically modified” refer to any chemical modification that does not involve nucleic acid elongation by chemical synthesis and/or enzymatic catalysis.

The chemical modification, according to the present invention, comprises introduction of a side chain terminating with a functional group to one or more purine or pyrimidine base(s) of the pre-existing nucleic acid. In other words, the chemical modification according to the present invention results in a functionallized pre-existing nucleic acid that includes one or more functional group(s) at the end of one or more side chain(s) that are covalently linked to one or more purine or pyrimidine base(s) of the pre-existing nucleic acid.

The side chain, according to the present invention, can be saturated or unsaturated. The side chain can include 2-20 carbon atoms, preferably 2-15 carbon atoms and, most preferably, 2-10 carbon atoms.

The side chain can be interrupted by one or more heteroatoms such as, but not limited to, O, S and N. The side chain can be further substituted by one or more chemical group such as, but not limited to, ═O, ═NH and an alkyl group having 1-3 carbon atoms.

According to a preferred embodiment, the method of the present invention can further include treating the pre-existing nucleic acid, prior to the chemical modification thereof, in order to restrict the chemical modification(s) imposed thereon to predetermined purine or pyrimidine bases. In other words, the method of the present invention can be directed toward chemically modifying the pre-existing nucleic acid at pre-determined, specific locations, which locations shall serve to covalently attach the pre-existing nucleic acid to a metal surface or cluster.

Thus, the pre-existing nucleic acid can be treated, prior to its chemical modification, so as to obtain a double-stranded nucleic acid that has one or more single-stranded regions at certain locations thereof. The resulting single-stranded endo- or exo-(overhang) regions include purine and/or pyrimidine bases, which are hence accessible to the chemical modification that follows, as opposed to purine or pyrimidine bases engaged in base pairing and which are protected from modification.

In one example of this embodiment of the present invention, a pre-existing double-stranded DNA is treated, prior to its chemical modification, by a sequence specific endonuclease (also known in the art as a restriction enzyme). This type of enzymatic restriction, which is also referred to herein as endonucleolysis, results in 3′ and/or 5′ single-stranded overhangs that include specific base sequences. The purine or pyrimidine bases in the resulting single stranded overhangs are highly accessible to the chemical modification that follows.

In another example of this embodiment of the present invention, a pre-existing double-stranded nucleic acid (DNA-DNA, DNA-RNA or RNA-RNA hybrids, for example) is treated, prior to its chemical modification, by a 3′ or 5′ specific exonuclease. Such treatment results in single-stranded overhangs that are accessible to the chemical modification that follows.

In another example, a pre-existing single-stranded nucleic acid (ssRNA or ssDNA) is protected by one or more complimentary oligonucleotides prior to its chemical modification. The complimentary oligonucleotide(s) is designed to produce double-stranded, and thus protected, nucleic acid at most regions, while leaving unprotected, and thus single-stranded, pre-determined regions of specific purine and/or pyrimidine bases in the pre-existing nucleic acid.

Following the chemical modification of the pre-existing nucleic acid, the obtained functionallized nucleic acid is interacted, according to this aspect of the present invention, with a metal surface, so as to covalently attach the pre-existing nucleic acid to the surface via the functional group. Thus, the pre-existing nucleic acid is attached to the metal surface through one or more side chains that are covalently linked at one end to a purine or pyrimidine base and at the other end to the metal surface.

The metal surface of the present invention is selected so as to enable the covalent attaching of the functional group thereto. Thus, the selected metal can be, for example, silver (Ag), gold (Au), mercury (Hg), platinum (Pt), molybdenum (Mo) and tungsten (W), preferably gold. The metal surface, according to the present invention, can be, for example, a metal plate, a metal film or a metal coat of another surface.

According to another preferred embodiment of the present invention, the gold surface is of a thin gold film, which has a thickness that ranges between 1 nm and 20 nm.

As described hereinabove, gold surfaces are recognized as highly advantageous nucleic acids supports since they are chemically inert to interactions with bio-macromolecules but, at the same time, accessible to chemisorption via the formation of stable, covalent gold-thiol bonds.

According to a presently preferred embodiment of the present invention, the covalent attachment of a pre-existing nucleic acid to a gold surface is effected via a thiol group introduced to the pre-existing nucleic acid. The obtained thiol-gold bonds are highly stable under variant conditions and therefore allow the use of such pre-existing nucleic acids covalently attached to a gold surface under variable conditions and in a variety of analyses.

Thus, according to a preferred embodiment of the present invention, the step of chemically modifying a pre-existing nucleic acid, as this term is defined hereinabove, comprises thiolating the pre-existing nucleic acid by introducing one or more side chain(s), as described hereinabove, that are terminated with a thiol group, to one or more purine or pyrimidine base(s) of the nucleic acid, so as to obtain a thiolated pre-existing nucleic acid.

As used herein the phrase “thiol group” refers to a —SH group.

Hence, the obtained functionallized pre-existing nucleic acid, according to this embodiment of the present invention, has one or more thiol groups, which serve as functional groups. Each thiol group is preferably, yet not obligatorily, present at the termini of the side chain that is covalently linked to a purine or pyrimidine base of the pre-existing nucleic acid.

The present invention thus further teach a method of thiolating a pre-existing nucleic acid, which method comprises covalently attaching a side chain terminating with a thiol group to one or more unpaired purine or pyrimidine bases of the pre-existing nucleic acid.

An unpaired purine base according to the present invention includes, for example, an adenine base or a guanine base, while an unpaired pyrimidine base includes, for example, a thymine base, a cytosine base or a uracil base. Unpaired bases can be obtained, for example, by the methods described hereinabove.

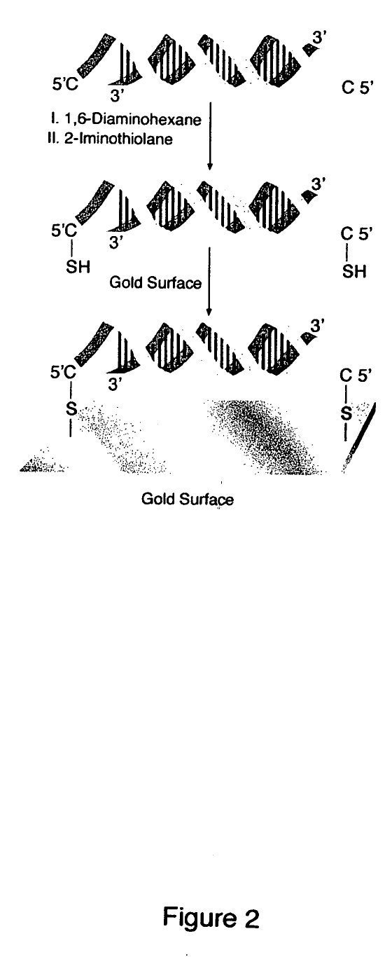

In a representative, yet non limiting, example, which is further detailed in the Examples section that follows and is schematically presented in FIG. 2, a double-stranded pre-existing DNA was enzymatically restricted so as to obtain two double-stranded fragments, where each fragment has a single-stranded overhang that includes an unpaired cytidine residue. Each fragment was thereafter reacted with 1,6-diaminohexane, so as to transaminate the unpaired cytidine residues and thereby obtain cytidine residues substituted by an aminohexane at the N⁴ positions thereof. The transaminated cytidine residues were thereafter reacted with 2-iminithiolane, so as to obtain a saturated side chain terminating with a thiol group, covalently attached to the N⁴ positions of each of the unpaired cytidine bases of the pre-existing DNA. The saturated side chain in this example had 10 carbon atoms, was interrupted by a nitrogen heteroatom and was substituted by a “═NH” chemical group.

The general methodology of the present invention, described hereinabove, is novel and highly advantageous in several respects:

(i) The method of the present invention provides a synthetic route to chemically modify a pre-existing nucleic acid, and thus enables the use of naturally occurring nucleic acids in nucleic acid chips;

(ii) The method of the present invention further provides a chemical modification of the pre-existing nucleic acid at pre-determined locations in the nucleic acids, and thus advantageously restricts the covalent attachment thereof to the metal surface to specific pre-selected locations. In particular, the present invention is highly advantageous since it enables to attach the pre-existing nucleic acid to the metal surface only through its ends and thus avoids adversely influencing its natural structure.

(iii) The method of the present invention further provides a controlled interaction between a pre-existing nucleic acid and the metal surface, which is effected by the presence of the side chain covalently linked to purine or pyrimidine bases. The presence of the side chain enables the attachment of the pre-existing nucleic acid to the metal surface without affecting its native structure, as described hereinabove. However, the length of the side chain can be controlled so as to either reduce interactions formed between the nucleic acid and the surface or to enable the performance of certain analyses that call for a short distance between the attached nucleic acid and the surface, as is further detailed hereinbelow.

(iv) The method of the present invention is highly versatile since it enables the use of a variety of functional groups, which can be covalently attached to a variety of metal surfaces and thus the method enables to control the nature and strength of the covalent attachment of the pre-existing nucleic acid to the metal surface.

(v) By covalently attaching the pre-existing nucleic acids to the metal surface through highly stable bonds, as in the example of thiol-gold bonds, a tight binding is achieved. This tight binding, along with the maintenance of the nucleic acid natural structure, as described above, enables reversible interaction between the covalently attached pre-existing nucleic acids with other macromolecules of choice, without affecting the binding of the attached nucleic acid. Thus, the pre-existing nucleic acid covalently attached to a metal surface by the method of the present invention is reusable.

Hence, according to another aspect of the present invention, there is provided a pre-existing nucleic acid covalently attached to a metal surface via the method described hereinabove.

Pre-existing nucleic acids covalently attached to a metal solid support can be manufactured according to the present invention in a microarray form, so as to provide a reusable nucleic acid chip. The nucleic acid chip of the present invention can serve to explore the structure and functionality of pre-existing nucleic acids and of macromolecular assemblies that include pre-existing nucleic acids. The nucleic acid chip of the present invention is novel and highly advantageous since it presents pre-existing nucleic acids (e.g., naturally occurring nucleic acids), as opposed to the presently used nucleic acid chips that typically present synthetically prepared nucleic acids, which are commonly obtained via in situ synthesis performed on the chip surface.

As has already been mentioned hereinabove, according to a preferred embodiment of the present invention, the pre-existing nucleic acids of the present invention, arranged, for example, in a microarray, can be interacted with macromolecules, preferably of a biological source, which is also referred to herein as a bio-macromolecule.

The bio-macromolecule can include, for example, a protein or a nucleic acid such as a RNA or DNA molecule.

The possibility to interact the pre-existing nucleic acid covalently attached to a metal surface with other macromolecules is attributed to the ability to attach the pre-existing nucleic acid to the metal surface without substantially interfering with its natural structure, as is further described hereinabove.

According to an embodiment of the present invention, the macromolecule that is interacted with the pre-existing nucleic acid attached to the metal surface is labeled with a detectable label. Labeling the macromolecule allows visualization of the macromolecular assembly obtained, using any one or a plurality of existing screening technologies.

Thus, according to another aspect of the present invention, there is provided a method of screening a nucleic acid chip presenting pre-existing nucleic acids covalently attached thereto and interacted with a macromolecule. The method according to this aspect of the present invention is effected by obtaining a nucleic acid chip presenting pre-existing nucleic acids covalently attached thereto as described hereinabove; interacting the pre-existing nucleic acids with macromolecules and screening the nucleic acid chip presenting the pre-existing nucleic acids covalently attached thereto for bound macromolecules.

Depending on the screening methodology employed, screening the nucleic acid chip described hereinabove can be performed directly or by labeling the macromolecules with a detectable label.

Direct screening of the nucleic acid chip can be performed, for example, using screening technologies such as AFM imaging, as is further detailed hereinbelow.

Other screening technologies, which typically require pre-labeling the macromolecules interacted with the pre-existing nucleic acid for better visualization and resolution, include, for example, fluorescence measurements (in which fluorescent/dye labels are employed), autoradiographing (in which radioactive labels are employed), transmission electron microscopy (TEM) scanning (in which electron dense labels are employed), dark-field scanning transmission electron microscopy (STEM), electron spectroscopic imaging (ESI), scanning tunneling microscopy (STM) and surface plasmon resonance spectroscopy (SPS).

Optionally, the macromolecules can be labeled by a metal cluster, as described for example by Medalia et al. (1999) and in WO 01/20017. The nucleic acid chip that presents such metal-tagged macromolecules can be screened using technologies such as, but not limited to, TEM, STEM or AFM imaging.

According to another aspect of the present invention, there is provided a method of imaging of a pre-existing nucleic acid. The method according to this aspect of the present invention is effected by obtaining a pre-existing nucleic acid covalently attached to a metal surface by the method described hereinabove and imaging the resulting immobilized pre-existing nucleic acid prior to and/or after its association with a macromolecule of choice.

According to a preferred embodiment of this aspect of the present invention, the imaging of the pre-existing nucleic acid is performed using, for example, AFM imaging.

As described hereinabove, AFM imaging has recently become a common and advantageous tool for structural and physical studies of biological macromolecules, mainly because it provides the ability to perform experiments with samples at their physiological environment. AFM measurements require the immobilization of a biological macromolecule to an inert surface. However, in order to achieve meaningful AFM imaging, the investigated macromolecule should be kept close enough to the surface, to enable its detection by the AFM tip. The pre-existing nucleic acids covalently attached to a metal surface according to the present invention are therefore highly suitable samples for efficient AFM imaging since, as described hereinabove, they are characterized by high adherence to the metal surface via one or more functional groups that are located at pre-determined locations in the pre-existing nucleic acids and by controlled distance from the surface, determined by the length of the side chains employed. It will be appreciated in this respect that all published AFM studies that involve nucleic acids covalently attached to a gold surface were performed using synthetically-prepared nucleic acids, while native nucleic acids and macromolecular assemblies including same have so far never been studied using AFM imaging.

As is further detailed in the Examples section that follows, clear and meaningful AFM images of pre-existing nucleic acids and of a representative macromolecular assembly including same were obtained using the method of the present invention.

However, other screening methods can be employed for imaging the pre-existing nucleic acid covalently attached to a metal surface of the present invention. Such screening methods include, for example, transmission electron microscopy (TEM) scanning, dark-field scanning transmission electron microscopy (STEM), electron spectroscopic imaging (ESI), surface plasmon resonance spectroscopy (SPS) and scanning tunneling microscopy (STM).

As is further described hereinabove, according to a preferred embodiment of the present invention, the metal surface employed is a gold film. Gold surfaces are known as suitable surfaces for imaging nucleic acids via AFM imaging, as well as other electronic scanning techniques. However, in order to achieve successful and meaningful AFM measurements, the supporting surface should be characterized by high flatness level. The presently known techniques for preparing gold surfaces suitable for AFM imaging are limited in this respect since they provide gold surfaces that are rough and/or contaminated. It will be appreciated that the use of such rough and/or contaminated gold surfaces is also limiting with respect to other applications.

In this respect, the present invention further provides a method of preparing a thin flat gold film that is devoid the limitations described hereinabove. The method according to this aspect of the present invention is effected by evaporating a metallic gold onto a chip under reduced pressure, while gradually heating the gold. According to a preferred embodiment of the present invention, the gold wire is evaporated onto a mica chip. According to another preferred embodiment of the present invention, the evaporation is effected under reduced pressure that ranges between 10⁻⁵ torr and 10⁻⁶ torr, preferably between 3×10⁻⁶ torr and 7×10⁻⁶ torr, and most preferably under reduced pressure that equals about 5×10⁻⁶ torr.

According to another preferred embodiment of the present invention, the method further comprises washing the gold film thus prepared, preferably with an organic solvent such as, but not limited to, dichloromethane. The washing step is preferably followed by drying the gold film, preferably in air. The washing and drying procedures are highly recommended since they stabilize the obtained film and enhance its hydrophobicity, which is highly advantageous in the context of the present invention, i.e., covalently attaching nucleic acids to the gold film while avoiding non-covalent interactions.

The gold films obtained by this method of the present invention are characterized by a thickness that ranges between 1 nm and 20 nm, preferably between 1 nm and 10 nm and most preferably between 4 nm and 6 nm. As is further detailed in the Examples section that follows, gold films that have a thickness of 4-6 nm are highly preferable since they are characterized by both stability under buffer conditions and reduced roughness. The gold films obtained by this method are further characterized by a mean roughness that ranges between 0.1 nm and 1 nm, preferably between 0.2 nm and 0.7 nm and most preferably between 0.2 nm and 0.5 nm. The thin gold films obtained by the method of the present invention are further characterized as transparent films and can therefore be further used as supports for optical measurements such as scanning near-field optical microscopy (SNOM).

The method of preparing thin flat gold films described herein is highly advantageous, also because of its simplicity.

The methods disclosed by the present invention are highly beneficial in the context of covalently attaching pre-existing nucleic acids to metal surfaces. However, the general methodology described herein, the gist thereof being chemical modification of pre-existing nucleic acid so as to render them reactive with metals, can similarly be used to covalently attach pre-existing nucleic acids to metal clusters.

Thus, according to another aspect of the present invention, there is provided a method of covalently attaching a pre-existing nucleic acid to a metal cluster. The method according to this aspect of the present invention is effected by chemically modifying the pre-existing nucleic acid, so as to obtain a functionallized pre-existing nucleic acid as described hereinabove, and attaching the functionallized pre-existing nucleic acid to a metal cluster, so as to obtain a pre-existing nucleic acid covalently attached to the metal cluster via one or more functional groups.

Thus, the chemically modifying step is effected as described hereinabove and the functionallized pre-existing nucleic acid obtained is thereafter reacted with a metal cluster, so as to obtain a covalently metal-tagged pre-existing nucleic acid.

The method of covalent metal-tagging pre-existing nucleic acids according to the present invention is highly beneficial since it enables the direct visualization of pre-existing nucleic acids and of macromolecular assemblies containing same by microscopical methods such as electron microscopy (EM) and AFM imaging.

This method is further advantageous over the presently known techniques of metal-tagging nucleic acids, since while the known methods teach metal-tagging of only synthetically-prepared nucleic acids, the present invention allows also metal-tagging of pre-existing, native, nucleic acids and thus enables to study, via microscopy, the structure of pre-existing nucleic acids and of macromolecular assemblies containing same, which is beneficial in elucidating their mode of action.

According to this method of the present invention, the metal cluster can include metals such as, but not limited to, Ag, Au, Hg, Pt, Mo and W. According to a preferred embodiment of the present invention, the metal cluster is a gold cluster. The gold cluster can include, for example, a maleimido derivative of a gold cluster, such as the commercially available “NANOGOLD” or colloidal gold of a pre-determined size, which can be prepared according to a procedure described hereinafter in the Examples section that follows.

The obtained metal-tagged pre-existing nucleic acid of the present invention can further interact with a macromolecule, preferably a bio-macromolecule, so as to form a metal-tagged macromolecular assembly including the pre-existing nucleic acid. The bio-macromolecule can be, for example, a protein or a nucleic acid. The bio-macromolecule can be labeled for enhanced visualization.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Materials and Methods

Preparation of gold films: An evaporation boat containing a piece of a metallic gold (1.0-2.5 mg) was placed in a vacuum evaporating system (BAE 080T, Balzers). A freshly cleaved mica chip was placed in a distance of about 10 cm from the evaporation boat, within the evaporation system. The pressure in the evaporation system was then reduced. When the vacuum reached a value below 5×10⁻⁶ torr, the boat was heated using electrical current. The shutter between the evaporation boat and the mica chip was opened after the gold had liquefied and the electrical current was increased gradually until the gold completely evaporated. The obtained gold plated mica chip was thereafter rinsed in dichloromethane for 30 seconds and dried in air. The gold film was kept at ambient conditions for 24 hours prior to its re-wetting and imaging.

Characterization of the gold films surfaces: The thickness of the gold film was measured using samples that were prepared by evaporating the gold, as described hereinabove, onto a mica surface that had been partially covered with 1 μm latex beads (Sigma). The plated mica chips were sonicated in the presence of dichloromethane for 30 seconds, so as to remove the beads and thus expose the mica surface as holes in the gold film (Fischer, 1998). AFM imaging of this area was then used to directly measure the gold film thickness. The RMS roughness of the gold films was calculated using the Nanoscope IIIa AFM software package (Digital Instrument Inc., Santa Barbara, Calif.).

Preparation of template stripped gold films: Gold was evaporated onto a freshly cleaved mica chip essentially as described by Hegner et al., 1993. Briefly, [111] textured gold substrates (Golan et al., 1992) were prepared by resistive evaporation of 100 nm gold wire (99.99% pure) onto freshly cleaved mica that had been pre-heated to 300° C. for 3 hours. After annealing for 3 hours at 250° C. in air, the gold surface was glued by cyanoacrylate glue to a glass cover slide and incubated for 12 hours at room temperature. The mica was mechanically stripped to reveal a flat gold surface that mimics the mica surface. The obtained gold surfaces were stored in a closed box.

Colloidal gold particles: The preparation of 3.5 nm colloidal gold was adapted from Slot and Geuze, 1985, with minor modifications. Eighty ml of 0.012% NaAuCl₄ in water and a 20 ml solution of 0.25% tannic acid, 0.2% sodium citrate and 1 mM potassium carbonate were each heated to 60° C. and rapidly mixed together. The gold colloids were formed within seconds. No additional purification was needed.

Introducing thiol-terminated linkers into a pre-existing nucleic acid—general approach: A nucleic acid is treated so as to generate one or more fragments which have one or more single-stranded regions that include unpaired bases. An unpaired base at the single-stranded region is chemically modified, so as to covalently attach thereto a side chain terminating with an amino group, which is thereafter thiolated to obtain nucleic acid molecules having thiol-terminated linkers.

Preparation of pre-existing DNA fragments having an unpaired cytidine base: (i) Thirty μg of a 3350-bp plasmid DNA [pT7-7 vector containing 873 bp insertion of the β subunit of the proteasome (Zuhl et al., 1997)] were digested with 60 units of XbaI (New England, Biolabs) for 3 h at 37° C. Fragments of 2450-bp and 900-bp, each having a 5′-CTAG overhang, were obtained. The fragments were purified by Gel Purification Kit (Qiagen). (ii) pBluescript (MBI Fermentas) was digested with Cfr10I to produce 1997-bp and 964-bp fragments having a 5′-CCGG overhang. The fragments were purified by Gel Purification Kit (Qiagen).

Thiolation of cytidine bases in pre-existing DNA fragments: The transamination of free cytidine bases with 1,6-diaminohexane was performed according to a procedure by Draper (Draper, 1984) with slight modifications. Three hundreds (300) μl of a transamination solution consisting of 3.7 M 1,6-hexanediamine, 1.2 M Na₂S₂O₅ and 1 mg of hydroquinone (pH 7.2) were added to a solution of the DNA restriction fragments, obtained as described hereinabove, in 40 μl of water. The mixture was incubated for 15 h at 42° C. The reaction mixture was then cooled on ice and the DNA was recovered by ethanol precipitation, followed by purification on a G-50 Quick Spin Column (Roche Diagnostics).

A solution of 200 μl of 0.5 M triethanolamine (BDH) (pH 8.4), 1 μl of 3 M NaOAc, 10 μl of 140 mM MgCl₂ and 1 mg of 2-iminothiolane-hydrochloride (Sigma) was added to 10 μg of the transaminated DNA, prepared as described hereinabove and dissolved in 100 μl of water. The resulting mixture was incubated for 6-10 h at 4° C. The DNA was recovered by ethanol precipitation, resuspended in 100 μl of purified water (Milli-Q Plus system) and further purified by a Gel Purification Kit (Qiagen).

Thiolation of adenosine bases in pre-existing DNA fragments: A pre-existing DNA is enzymatically digested to obtain pre-existing DNA fragments having overhangs that include one or more unpaired adenosine base(s). The conversion of a free adenosine base in the obtained DNA restriction fragments to N⁶-(carboxymethyl)adenosine is performed according to Gebeyehu et al. (1987). A mixture of the DNA restriction fragment, sodium iodoacetate and water at pH=6.5 is stirred for four days at 30° C. The reaction mixture is then cooled, NaCl is addedd to 0.5 M, and the DNA is recovered by precipitation with 2.5 volumes of cold (−20° C.) ethanol. The DNA is further purified by a Gel Purification Kit (Qiagen).

The obtained DNA fragments having a N⁶-(carboxymethyl)adenosine overhang is thereafter reacted with 1,6,-diaminohexane, so as to obtain a DNA fragment having a N⁶-[(6-aminohexyl)carbamoylmethyl]adenosine overhang. The DNA fragment having a N⁶-(carboxymethyl)adenosine is dissolved in a 1 M aqueous solution of 1,6 diaminohexane, which is adjusted to pH 4.7 with 5 M HCl. Ethyldimethylaminopropyl carbodiimide (EDC) is added and the mixture is stirred for 2 hours. Two additional portions of EDC are added at 30 minutes intervals. The DNA is recovered by ethanol precipitation, resuspended in 100 μl of purified water (Milli-Q Plus system) and further purified by a Gel Purification Kit (Qiagen).

Thiolation of a DNA fragment having an overhang that include N⁶-[(6-aminohexyl)carbamoylmethyl]adenosine with 2-iminothiolane is carried out as described above for cytidine bases.

Thiolation of thymidine-guanosine and uracil bases in pre-existing DNA fragments: The examples hereinabove describe the thiolation of cytidine, and adenosine bases in pre-existing DNA. It would be appreciated by one ordinarily skilled in the art, that similar or somewhat modified procedures could be applied to thiolate unpaired thymidine and guanosine bases in pre-existing DNA fragments. It would also be appreciated by one ordinarily skilled in the art, that similar or somewhat modified procedures could be applied to thiolate unpaired uracil bases in the rare occasions that such occur in pre-existing DNA fragments.

Thiolation of terminal cytidines in oligodeoxynucleotides: Terminally thiolated oligodeoxynucleotides were prepared using analogous procedures.

The preparation of thiolated single-stranded oligodeoxynucleotides containing a single terminal cytidine was applied using the procedure described hereinabove.

Single-stranded oligodeoxynucleotides containing more than one cytidine base were hybridized to complementary oligonucleotides, leaving a single stranded cytidine-containing overhang, and were thiolated thereafter according to the following procedure:

To a mixture of 8 ml concentrated HCl and 7 ml DDW, 4 ml ethylendiamine were added. The mixture was cooled on ice and 3.7 grams of Na₂S₂O₅ were then added very slowly with constant stirring. The pH was adjusted to 7.2 with a solution of 10 M NaOH (about 600 μl). Nine ml of the obtained solution were thereafter mixed with 1 ml solution of about 700 nmol of a cytidine-terminated synthetic oligodeoxynucleotide in water. A solution of 15 mg hydroquinone dissolved in a minimum volume of absolute ethanol was then added and the mixture was incubated for 15 hours at 42° C. The obtained reaction mixture was thereafter dialyzed against DDW (membrane cut-off 1200; Sigma D-7884) and lyophilized. The resulting powder was dissolved in about 0.5 ml DDW, 0.3 ml 2 M triethanolamine (pH 8.5) and 2 mg of 2-iminothiolane-HCl were added and the mixture was incubated for 12 hours at 4° C. The obtained reaction mixture was dialyzed against DDW as described above and lyophilized.

Covalently attaching pre-existing DNA to gold films: A 10 μl droplet containing about 500 ng of unmodified or thiolated DNA was deposited on a gold film for 1-2 hours. The sample was washed by three sequential 1:1 dilutions with water (Milli-Q Plus system) and imaged in the AFM apparatus without any drying steps.

Covalently attaching terminally thiolated DNA oligonucleotides to colloidal gold: An aqueous suspension of 10¹²-10¹³ 20 nm colloidal gold particles, prepared as described hereinabove, in 0.5 ml of water was mixed with 20 μl of 0.5 mM solution of terminally-thiolated oligodeoxynucleotide, prepared as described hereinabove. The mixture was incubated at 4° C. for 12 hours. The gold-oligodeoxynucleotide conjugate was recovered by centrifugation.

Covalently attaching thiolated DNA to colloidal gold: An aqueous suspension of 10¹²-10¹³ 20 nm colloidal gold particles, prepared as described hereinabove, in 0.5 ml of water is mixed with 20 μl of 0.5 mM solution of terminally-thiolated pre-existing DNA fragments, prepared as described hereinabove. The mixture is incubated at 4° C. for 12 hours. The gold-DNA conjugates are recovered by ethanol precipitation.

Preparation of SspC-DNA complexes: Small spore protein C (SspC) from B. Subtillis was expressed in E. Coli and purified as described by Nicholson et al. Three μl of 1 μg/ml unmodified or thiolated DNA in 10 mM Tris-maleate, pH 6.9, were added to 97 μl of the same buffer containing 16.5 ng of the purified protein, to obtain a SspC-DNA complex.

Binding of SspC-DNA Complexes to Gold Films:

a. Direct deposition: A 20-μl droplet containing pre-formed SspC-DNA complexes in 10 mM Tris-maleate, pH 6.9, was deposited on the gold film as described hereinabove and washed by three sequential 1:1 dilutions with the same buffer.

b. Complex formed on the gold surface: A 20-μl droplet containing 0.6 ng of thiolated DNA fragments was deposited on the gold film and incubated for 30 minutes. After washing with Tris-maleate buffer, 3 ng of SspC in 10 μl of the same buffer were added, incubated for another 30 minutes and washed.

AFM measurements and image processing: All AFM measurements were carried out using a Nanoscope IIIa atomic force microscope with a multimode head (Digital Instruments Inc., Santa Barbara, Calif.). Images of the DNA-gold samples were recorded in solution in a glass fluid cell (Digital Instruments Inc., Santa Barbara, Calif.) operating in tapping mode. Oxide sharpened NPS-10 cantilevers (Digital Instruments Inc., Santa Barbara, Calif.) were operated close to their resonance frequency (8-10 kHz) in the aqueous solution. Images of the gold films were taken in air, using microfabricated cantilevers (Silicon-MDT) with resonance frequency of about 300 kHz. All AFM images of DNA preparations were processed by high-pass filtering to enhance the contours of DNA and to suppress long-range height variations in the support.

### Experimental Results

Thin gold films: The gold films prepared by the method of the present invention described hereinabove, were found to be highly advantageous over presently known gold films in several aspects. The major advantage of the method is in its simplicity. The resulting gold films can be used directly after the evaporation step, and do not require further processing that could damage their surfaces (e.g., detachment from the mica support). Furthermore, the flatness of the supporting gold surface, which is a key issue for a successful and meaningful AFM experiments, was achieved using a simple and conventional evaporating system. By avoiding experimental procedures such as heating the mica surfaces or annealing the gold after deposition, the obtained gold films were characterized by flat surfaces and thus by clear AFM images obtained using same. Moreover, upon exposure to air, the films' surfaces obtained were highly hydrophobic and were therefore readily available for the specific binding of thiolated macromolecules, with minimized non-specific binding, as is further detailed hereinbelow.

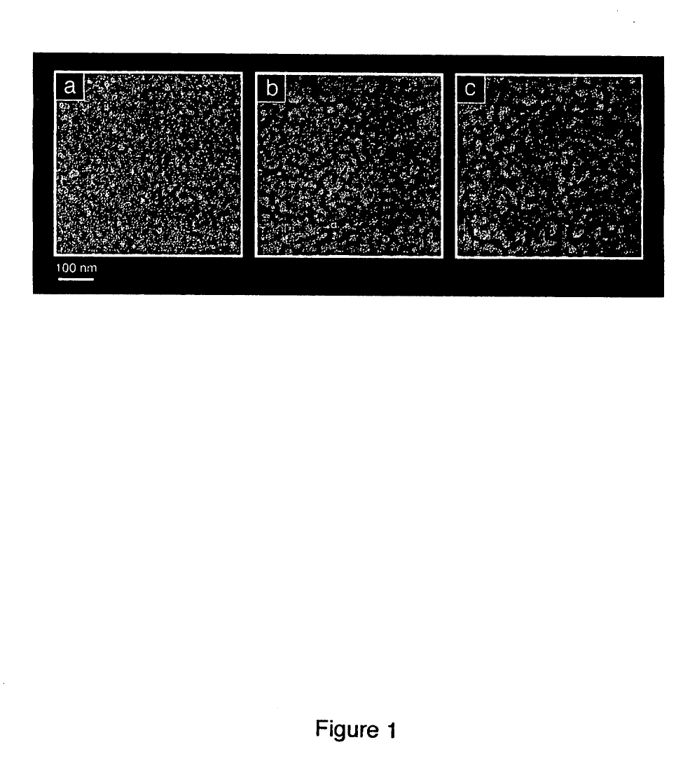

The thickness of the films was measured by partially covering the mica chip with latex beads that were removed after the gold had been evaporated, leaving free mica surfaces. Thereby, the height of the gold films could be directly measured by a conventional tapping mode AFM experiment. Using this approach, a set of gold surfaces having a thickness ranging between 1 nm and nm were produced. Each of the obtained surfaces was characterized by AFM imaging in ambient conditions. FIG. 1 presents three typical gold surfaces, 0.25 μm² each, having a thickness of 1 nm (FIG. 1a), 6 nm (FIG. 1b) and 10 nm (FIG. 1c). The mean roughness (RMS) of the surfaces of these samples was calculated from an area of 4 μm² and was found to be 0.2 nm, 0.5 nm and 0.7 nm, respectively. The surface roughness of gold films having a thickness of 6 nm and less is similar to the surface roughness of films prepared by the TSG technique (0.3 nm), which is frequently used in AFM studies (Hegner et al., 1993; Thomson et al., 1999). However, the films having a thickness of 1 nm were found somewhat unstable for TMAFM experiments in solution, although the roughness thereof was relatively low. Contrary, the 4-6 nm films were found stable under buffer conditions for many hours, as long as they remained wet, and can therefore serve as highly preferable supports for AFM experiments in solution.

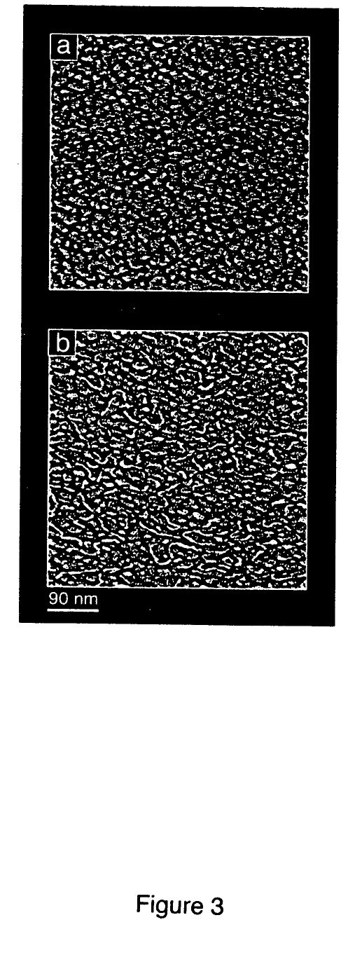

AFM imaging of pre-existing DNA molecules attached to thin gold films: Incubating a solution of thiolated pre-existing DNA and the gold films prepared as described hereinabove resulted in covalent attachment of the DNA to the flat gold surfaces and enabled its imaging by AFM with no intermediate step of sample drying. The AFM images of unmodified DNA and thiolated DNA upon incubation with gold films having a thickness of 4-6 nm are presented in FIGS. 3a and 3b, respectively. The obtained images suggest that the DNA attachment to the gold surfaces occurred through the terminal thiol groups since the surface image of a gold film that was incubated with the unmodified DNA (FIG. 3a) does not differ from the image of an untreated surface (FIG. 1). This observation further indicates that the unmodified DNA fragments do not attach to the surface due to the hydrophobic nature of the gold surface. Following the incubation with thiolated DNA, the AFM image (FIG. 3b) shows ‘worm-like’ features on top of the gold surface. These features are attributed to DNA molecules that were attached to the gold surface through their thiolated linkers.

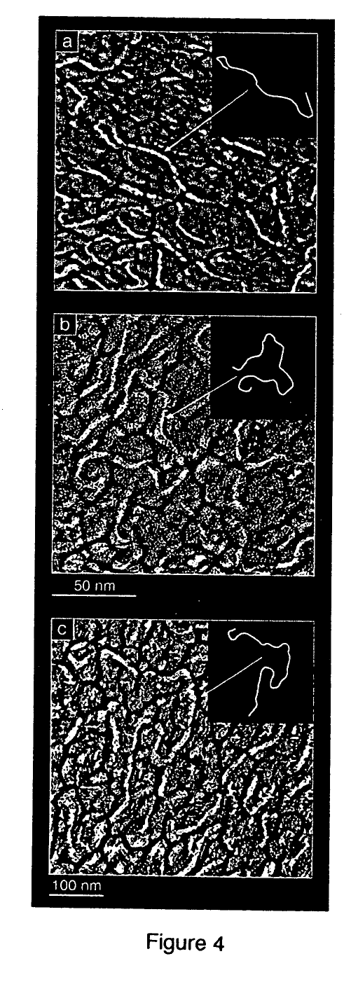

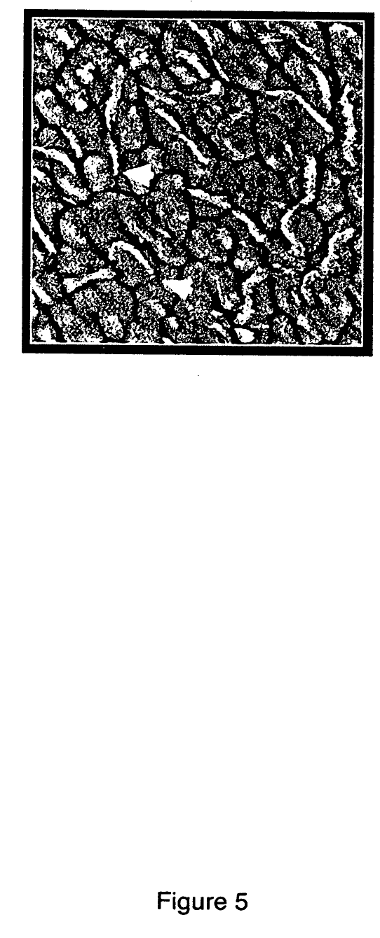

As is shown in FIGS. 4 and 5, a hardware zoom-in that enabled the imaging of individual DNA molecules was performed, in order to further characterize the surface-attached DNA. FIG. 4 shows several samples where tracing the contour length of the DNA revealed molecules of about 300 nm (FIGS. 4a and 4b) and about 700 nm (FIG. 4c), which represent the respective 900 bp and 2450 bp fragments that were chemisorbed to the surface.

However, as shown in FIG. 5, most of the visualized DNA corresponded to shorter molecules, although the input DNA contained only fragments of 900 bp and 2450 bp. One class of these molecules was characterized by thin lines of about 5 nm in width at the beginning, whose height above the surface is about 1.5-2.5 nm, as marked by the arrows in FIG. 5 (which agrees with the value of 2.2 nm for the diameter of B-form helical DNA), which become wider as the distance from the end of the DNA duplex grows, until they completely disappear and occasionally reappear after a short distance. The second class consists of molecules of 10-20 nm width. These molecules were assigned as fragments that are-middle parts of the whole DNA molecule. The phenomenon of widening and disappearance of the molecules is attributed to the fact that the site of its anchoring to the surface is located at the ends of the whole DNA molecules. It can thus be suggested that while the DNA molecules are tightly bound to the surface at their ends, the interaction of the middle parts of the molecules with the surface is lower due to the hydrophobic nature of the gold surface. Therefore, these parts of the molecules are nearly free to move in solution and appear either as a thick line or cannot be detected at all by the AFM tip. In these locations, the interaction of the AFM tip with the molecules is at least stronger then the interaction between the DNA and the surface. Due to this substantially reduced interaction between the DNA and the support and the fact that the DNA was kept hydrated throughout all manipulations, it is presumed that the native conformation of the DNA did not change during the described manipulations. Thus, the resulting gold-attached DNA should be readily available for further interactions of physiological significance, as detailed hereinbelow.

AFM imaging of SspC-DNA complex attached to a gold film: The feasibility of the method of the present invention was further demonstrated by studying an SspC-DNA complex attached to a gold surface, by TMAFM measurements in solution. SspC is a member of the SASP (small, acid-soluble spore proteins) family, which includes proteins that are involved in the condensation of the bacterial DNA into spores and protecting it from dry and wet heat, UV and gamma radiation, extreme desiccation (including vacuum) and oxidizing agents (Setlow, 1995). A unique feature of the DNA in dormant spores is that the spore chromosome is saturated with a group α/β-type SASPs (Setlow et al., 1992). The SASPs are small proteins of 68-71 residues with molecular mass of about 7 kDa. The sequence of these proteins is highly conserved both within a single species and across various Bacillus species. In vitro studies have shown that the SASPs are nonspecific double-stranded DNA-binding proteins and do not bind single-stranded DNA and single-stranded or double-stranded RNA (Setlow et al., 1992). The stochiometry of the SASP-DNA binding is 1 protein molecule per 4-5 bp of DNA, which is close to the ratio of α/β-type SASP-DNA found in spores. Hence, these proteins bind to all natural double-stranded DNA molecules and saturate them completely. The experiments were conducted with SspC as a representative SASP since negatively stained SspC-DNA complexes were visualized by transmission electron microscopy (Griffith et al., 1994).

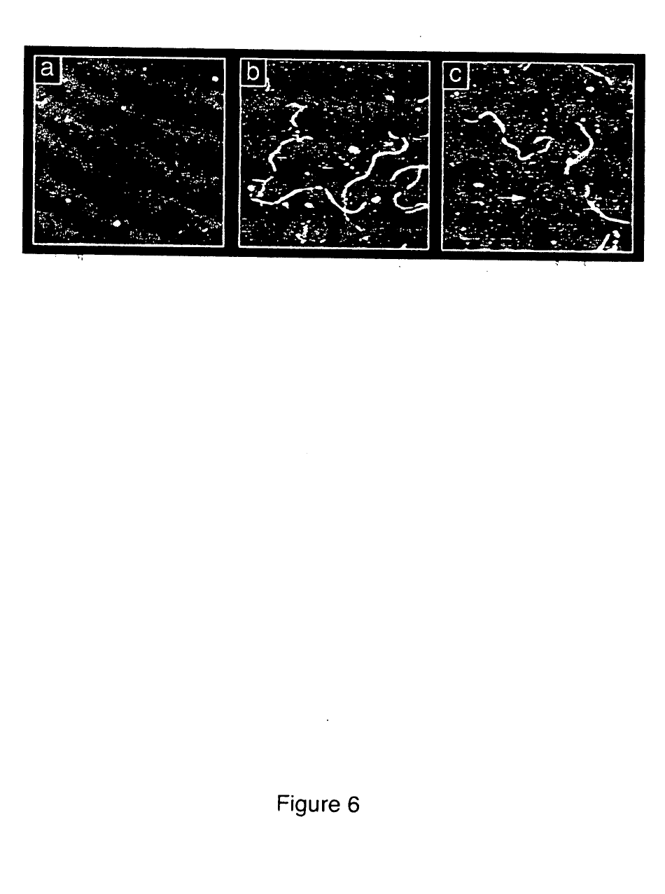

SspC-DNA surface-attached complexes were thus formed via two approaches: In one type of experiments, terminally thiolated DNA was first attached to the gold surface, unbound DNA was washed away, and the attached DNA was reacted with SspC protein. In the second type of experiments, the SspC-DNA complex was first formed in a test tube and was then applied to the gold surface and washed with buffer. As shown in FIG. 6, AFM images of both the complex formed with pre-attached DNA (FIG. 6b) and the attached pre-formed complex (FIG. 6c) can be visualized, while the complexes prepared on a surface that was treated either with unmodified DNA or with a pre-formed SspC-unmodified DNA complex, could not be detected (FIG. 6a). Furthermore, it is shown that although the protein-DNA complexes were prepared in the presence of a large excess of SspC, the adsorption of the free protein to the surface was not detected. The diameter of the SspC-DNA complex was found to be 4.5±0.5 nm, whether measured for the surface-formed complex (FIG. 6b) or for the pre-formed complex (FIG. 6c). This value is significantly close to 5.0±0.5 nm, which is the value obtained by transmission electron microscopy measurements of the negatively stained SspC-DNA complexes (A. Minski and E. Shimoni, personal communication). This successful formation of the protein-DNA complex in the immobilized state is attributed to the fact that the DNA was covalently attached to the surface only through its ends, thus allowing the rest of the molecule to make biologically significant interactions with a protein, and yet keeping the complex close enough to the surface to be imaged by the AFM tip. These results are also consistent with the notion that the binding of SspC to DNA is cooperative (Griffith et al., 1994). As shown in FIG. 6c, only fully protein-covered or completely naked DNA molecules, as marked by the arrow, can be detected after several washing steps that dissociate the complex.

The present invention discloses novel methods of chemically modifying a pre-existing nucleic acid, so as to obtain a functionallized pre-existing nucleic acid having a side chain terminating with a functional group covalently attached thereto. The obtained functionallized pre-existing nucleic acid can be covalently attached to a metal surface or a metal cluster, and can thereafter further interact with a macromolecule to form a macromolecular assembly.

As a model approach, thiolation of a pre-existing DNA and covalent attachment of the thiolated macromolecule to a gold surface was chosen. The obtained pre-existing DNA covalently attached to the gold surface through the thiol groups was successfully imaged by AFM per se and upon interacting thereof with a protein.

However, it would be appreciated by one ordinarily skilled in the art, that the methodology disclosed herein could potentially be applied to other pre-existing nucleic acids and to a large variety of macromolecular assemblies.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED

Alivisatos, A. P., Johnsson, K. P., Peng, X., Wilson, T. E., Loweth, C. J., Bruchez, M. J., and Schultz, P. G. (1996). Organization of ‘nanocrystal molecules’ using DNA. Nature 382, 609-611.

Boisset, N., Grassucci, R., Penczek, P., Delain, E., Pochon, F., Frank, J., and Lamy, J. N. (1992). Three-dimensional reconstruction of a complex of human alpha 2-macroglobulin with monomaleimido Nanogold (Au1.4 nm) embedded in ice. J. Struct. Biol. 109, 39-45.

Brockman, J. M., Frutos, A. G., and Corn, R. M. (1999). A multistep chemical modification procedure to create DNA arrays on gold surfaces for the study of protein-DNA interactions with surface plasmon resonance imaging. Journal of the American Chemical Society 121, 8044-8051.

Burry, R. W., Vandre, D. D., and Hayes, D. M. (1992). Silver enhancement of gold antibody probes in pre-embedding electron microscopic immunocytochemistry. J. Histochem. Cytochem. 40, 1849-1856.

Cheung, V. G., Morley, M., Aguilar, F., Massimi, A., Kucherlapati, R., and Childs, G. (1999). Making and reading microarrays. Nat. Genet. 21 (Suppl), 15-19.

Delamarche, E., Sundarababu, G., Biebuyck, H., Michel, B., Gerber, C., Sigrist, H., Wolf, H., Ringsdorf, H., Xanthopoulos, N. & Mathieu, H. J. (1996) Langmuir 12, 1997-2006.

Drake, B., Prater, C. B., Weisenhorn, A. L., Gould, S. A., Albrecht, T. R., Quate, C. F., Cannell, D. S., Hansma H. G. & Hansma P. K. (1989) Science 243, 1586-9.

Draper, D. E. (1984), Attachment of reporter groups to specific, selected cytidine residues in RNA using a bisulfite-catalyzed transamination reaction, Nucleic Acids Res. 12, 989-1002.

Engel, A., Lyubchenko, Y. & Muller, D., (1999) Trends Cell Biol. 9, 77-80.

Finklea, H. O. (1996), Electrochemistry of organized monolayers of thiols and related molecules on electrodes. In Electroanalytical Chemistry: a Series of Advances, Vol. 19, ed. (New York: Marcel Dekker), Vol. 19, pp. 109-335.

Fischer, U. C. (1998), Latex-Projection Patterns. In Procedures in Scanning Probe Microscopies, al., C. e., ed. Wiley & Sons), pp. 10^{-11.}

Gebeyehu, G., Rao, P. Y., SooChan, P., Simms, D. A., and Klevan, L. (1987). Novel biotinylated nucleotide—analogs for labeling and colorimetric detection of DNA. Nucleic Acids Res. 15, 4513-4534. Ghosh, S. S., Kao, P. M., McCue, A. W., and Chappelle, H. L. (1990). Use of maleimide-thiol coupling chemistry for efficient syntheses of oligonucleotide-enzyme conjugate hybridization probes. Bioconjug Chem 1, 71-76.

Golan, Y., Margulis, L., and Rubinstein, I. (1992), Vacuum-deposited gold films I. Factors affecting the film morphology. Surface Science 264, 312-326.

Goodwin, J. T., and Glick, G. D. (1993). Incorporation of Alkylthiol Chains at C-5 of Deoxyuridine. Tetrahedron Lett 34, 5549-5552.

Griffith, J., Makhov, A., Santiago-Lara, L., Setlow, P. (1994) Proc. Natl. Acad. Sci. USA 91, 8224-8.

Guthold, M., Zhu, X., Rivetti, C., Yang, G., Thomson, N. H., Kasas S., Hansma, H. G., Smith, B., Hansma, P. K. & Bustamarite, C. (1999) Biophys J 77, 2284-94.

Hainfeld, J. F., and Furuya, F. R. (1992). A 1.4-nm gold cluster covalently attached to antibodies improves immunolabeling. J. Histochem. Cytochem. 40, 177-184.

Hanna, M. M., Dissinger, S., Williams, B. D., and Colston, J. E. (1989). Synthesis and characterization of 5-[(4-Azidophenacyl)thio]uridine 5′-triphosphate, a cleavable photo-cross-linking nucleotide analogue. Biochemistry 28, 5814-5820.

Hanna, M. M. Zhang, Y., Reidling, J. C., Thomas, M. J., and Jou, J. (1993). Synthesis and characterization of a new photocrosslinking CTP analog and its use in photoaffinity labeling E. coli and T7 RNA polymerases. Nucleic Acids Res. 21, 2073-2079.

He, L., Musick, M. D., Nicewarner, S. R., Salinas, F. G., Benkovic, S. J., Natan, M. J., and Keating, C. D. (2000). Colloidal Au-enhanced surface plasmon resonance for ultrasensitive detection of DNA hybridization. Journal of the American Chemical Society 122, 9071-9077.

Hegner, M., Wagner, P., and Semenza, G. (1993). Immobilizing DNA on gold via thiol modification for atomic force microscopy imaging in buffer solutions. FEBS Lett. 336, 452456.

Hegner, M., Wagner, P., and Semenza, G. (1993). Ultralarge Atomically Flat Template-Stripped Au Surfaces for Scanning Probe Microscopy. Surface Sci. 291, 39-46.

Jordan, C. E., Frutos, A. G., Thiel, A. J., and Corn, R. M. (1997). Surface plasmon resonance imaging measurements of DNA hybridization adsorption and streptavidin/DNA multilayer formation at chemically modified gold surfaces. Analytical Chemistry 69, 4939-4947.

Kelley, S. O., Barton, J. K., Jackson, N. M., McPherson, L. D., Potter, A. B., Spain, E. M., Allen, M. J., and Hill, M. G. (1998). Orienting DNA helices on gold using applied electric fields. Langmuir 14, 6781-6784.

Knarr, R. F., Quon, R. A., and Vanderlick, T. K. (1998). Direct force measurements at the smooth gold/mica interface. Langmuir 14, 6414-6418.

Langer, P. R., Waldrop, A. A., and Ward, D. C. (1981). Enzymatic synthesis of biotin-labeled polynucleotides: Novel nucleic acid affinity probes. Proc. Natl. Acad. Sci. USA 78, 6633-6637. Medalia, O., Heim, M., Guckenberger, R., Sperling, R., and Sperling, J. (1999). Gold-Tagged RNA-A Probe for Macromolecular Assemblies. J. Struct. Biol. 127, 113-119.

Nicholson, W. O., Setlow B. and Setlow P. (1990), J. Bacteriol. 172, 6900-6.

Rief, M., Clausen-Schaumann, H., and Gaub, H. E. (1999). Sequence-dependent mechanics of single DNA molecules. Nat. Struct. Biol. 6, 346-349.

Sabatani, E., and Rubinstein, I. (1987). Organized Self-Assembling Monolayers on Electrodes 0.2. Monolayer-Based Ultramicroelectrodes For the Study of Very Rapid Electrode-Kinetics. J. of Phys. Chem. 91, 6663-6669.

Safer, D., Bolinger, L., and Leigh, J. S. J. (1986). Undecagold clusters for site-specific labeling of biological macromolecules: Simplified preparation and model applications. J. Inorg. Biochem. 26, 77-91.

Samori, P., Diebel, J., Lowe, H., and Rabe, J. P. (1999). Template-stripped gold supported on Ni as a substrate for SAMs. Langmuir 15, 2592-2594.

Setlow B., Sun, D., Setlow, P. (1992) J Bacteriol 174, 2312-22.

Setlow P. (1995) Annu Rev Microbio 49, 29-54.

Sibbald, M. J., Carlemalm, E. C., Beer, M., and Sproat, B. S. (1993). Imaging of RNA-protein interactions in splicing complexes with dark-field STEM. J. Struct. Biol. 110, 111-121.

Slot, J. W., and Geuze, H. J. (1985). A new method of preparing gold probes for multiple-labeling cytochemistry. Eur. J. Cell Biol. 38, 87-93.

Southern, E., Mir, K., and Shchepinov, M. (1999). Molecular interactions on microarrays. Nat. Genet. 21 (Suppl), 5-9.

Steel, A. B., Levicky, R. L., Herne, T. M., and Tarlov, M. J. (2000). Immobilization of nucleic acids at solid surfaces: Effect of oligonucleotide length on layer assembly. Biophysical Journal 79, 975-981.

Thomson, N. H., Smith, B. L., Almqvist, N., Schmitt, L., Kashlev, M., Kool, E. T. & Hansma, P. K. (1999) Biophys. J. 76, 1024-33.

Ulman, A. (1996). Formation and structure of self-assembled monolayers. Chemical Reviews 96, 1533-1554.

Vaish, N. K., Fraley, A. W., Szostak, J. W., and McLaughlin, L. W. (2000). Expanding the structural and functional diversity of RNA: analog uridine triphosphates as candidates for in vitro selection of nucleic acids [In Process Citation]. Nucleic Acids Res. 28, 3316-3322.

Yang, M. S., Yau, H. C. M., and Chan, H. L. (1998). Adsorption kinetics and ligand-binding properties of thiol-modified double-stranded DNA on-a gold surface. Langmuir 14, 6121-6129.

Zuhl, F., Tamura, T., Dolenc, I., Cejka, Z., Nagy, I., De Mot, R., and Baumeister, W. (1997). Subunit topology of the Rhodococcus proteasome. FEBS Lett 400, 83-90.

1. A method of covalently attaching a pre-existing nucleic acid to a metal surface, the method comprising:
chemically modifying at least one purine or pyrimidine base of said pre-existing nucleic acid by covalently linking to said at least one purine or pyrimidine base at least one side chain terminating with at least one functional group, so as to obtain a functionallized pre-existing nucleic acid having said at least one functional group at a termini of said at least one side chain, said at least one side chain being covalently linked to said at least one purine or pyrimidine base of said pre-existing nucleic acid; and
attaching said functionallized pre-existing nucleic acid to a metal surface, so as to covalently attach said pre-existing nucleic acid to said metal surface via said at least one functional group.

2. The method of claim 1, wherein said pre-existing nucleic acid is RNA.

3. The method of claim 1, wherein said pre-existing nucleic acid is DNA.

4. The method of claim 1, wherein said pre-existing nucleic acid includes at least one base analog.

5. The method of claim 1, wherein said pre-existing nucleic acid is treated prior to said step of chemically modifying, so as to restrict chemical modifications to predetermined purine or pyrimidine bases in said pre-existing nucleic acid.

6. The method of claim 5, wherein said pre-existing nucleic acid is a double stranded DNA and is treated by a sequence specific endonuclease prior to said step of chemically modifying, so as to restrict chemical modifications to predetermined purine or pyrimidine bases in a 3′ or 5′ single stranded overhang generated by endonucleolysis.

7. The method of claim 5, wherein said pre-existing nucleic acid is a double stranded nucleic acid and is treated by a 3′ or 5′ specific exonuclease prior to said step of chemically modifying so as to restrict chemical modifications to predetermined purine or pyrimidine bases in a 5′ or 3′ single stranded overhang generated by said 3′ or 5′ specific exonucleolysis, respectively.

8. The method of claim 5, wherein said pre-existing nucleic acid is a single stranded nucleic acid and is treated by at least one complementary protecting polynucleotide prior to said step of chemically modifying so as to restrict chemical modifications to predetermined purine or pyrimidine bases in at least one region not protected by said at least one complementary protecting polynucleotide, following hybridization with said at least one complementary protecting polynucleotide.

9. The method of claim 1, wherein said chemically modifying comprises thiolating said at least one purine or pyrimidine base of said pre-existing nucleic acid.

10. The method of claim 1, wherein said functional group is a thiol group.

11. The method of claim 1, wherein said step of chemically modifying comprises thiolating said at least one purine or pyrimidine base of said pre-existing nucleic acid, so as to obtain a thiolated pre-existing nucleic acid having a thiol functional group at said termini of said side chain being covalently linked to said at least one purine or pyrimidine base of said nucleic acid.

12. The method of claim 1, wherein said side chain is saturated or unsaturated and has 2-20 carbon atoms.

13. The method of claim 12, wherein said saturated or unsaturated side chain has 2-10 carbon atoms.

14. The method of claim 12, wherein said saturated or unsaturated side chain is interrupted by at least one heteroatom selected from the group consisting of O, S and N and/or is substituted by at least one chemical group selected from the group consisting of ═O, ═NH and an alkyl group having 1-3 carbon atoms.

15. The method of claim 1, wherein said metal surface is selected from the group consisting of a metal plate, a metal film and a metal coat.

16. The method of claim 1, wherein said metal is selected from the group consisting of Ag, Au, Hg, Pt, Mo and W.

17. The method of claim 15, wherein said metal is gold.

18. The method of claim 17, wherein said metal film is a thin gold film having a thickness ranging between 1 nm and 20 nm.

19. A pre-existing nucleic acid covalently attached to a metal surface by the method of claim 1.

20. The pre-existing nucleic acid of claim 19, interacted with a macromolecule.

21. The pre-existing nucleic acid of claim 20, wherein said macromolecule is labeled.

22. The pre-existing nucleic acid of claim 20, wherein said macromolecule is of a biological source.

23. The pre-existing nucleic acid of claim 20, wherein said macromolecule is a nucleic acid.

24. The pre-existing nucleic acid of claim 20, wherein said macromolecule is a protein.

25. The pre-existing nucleic acid of claim 19, wherein said pre-existing nucleic acid is RNA.

26. The pre-existing nucleic acid of claim 19, wherein said pre-existing nucleic acid is DNA.

27. The pre-existing nucleic acid of claim 19, wherein said pre-existing nucleic acid includes at least one base analog.

28. The pre-existing nucleic acid of claim 19, wherein said pre-existing nucleic acid is treated prior to said step of chemically modifying so as to restrict chemical modifications to predetermined purine or pyrimidine bases in said pre-existing nucleic acid.

29. The pre-existing nucleic acid of claim 28, wherein said pre-existing nucleic acid is a double stranded DNA and is treated by a sequence specific endonuclease prior to said step of chemically modifying, so as to restrict chemical modifications to predetermined purine or pyrimidine bases in a 3′ or 5′ single stranded overhang generated by endonucleolysis.

30. The pre-existing nucleic acid of claim 28, wherein said pre-existing nucleic acid is a double stranded nucleic acid and is treated by a 3′ or 5′ specific exonuclease prior to said step of chemically modifying so as to restrict chemical modifications to predetermined purine or pyrimidine bases in a 5′ or 3′ single stranded overhang generated by said 3′ or 5′ specific exonucleolysis, respectively.

31. The pre-existing nucleic acid of claim 28, wherein said pre-existing nucleic acid is a single stranded nucleic acid and is treated by at least one complementary protecting polynucleotide prior to said step of chemically modifying so as to restrict chemical modifications to predetermined purine or pyrimidine bases in at least one region not protected by said at least one complementary protecting polynucleotide, following hybridization with said at least one complementary protecting polynucleotide.

32. The pre-existing nucleic acid of claim 19, wherein said chemically modifying comprises thiolating said at least one purine or pyrimidine base of said pre-existing nucleic acid.

33. The pre-existing nucleic acid of claim 19, wherein said functional group is a thiol group.

34. The pre-existing nucleic acid of claim 19, wherein said step of chemically modifying comprises thiolating said at least one purine or pyrimidine base of said pre-existing nucleic acid, so as to obtain a thiolated pre-existing nucleic acid having a thiol functional group at said termini of said side chain being covalently linked to said at least one purine or pyrimidine base of said nucleic acid.

35. The pre-existing nucleic acid of claim 19, wherein said side chain is saturated or unsaturated and has 2-20 carbon atoms.

36. The pre-existing nucleic acid of claim 35, wherein said saturated or unsaturated side chain has 2-10 carbon atoms.

37. The pre-existing nucleic acid of claim 35, wherein said saturated or unsaturated side chain is interrupted by at least one heteroatom selected from the group consisting of O, S and N and/or is substituted by at least one chemical group selected from the group consisting of ═O, ═NH and an alkyl group having 1-3 carbon atoms.

38. The pre-existing nucleic acid of claim 19, wherein said metal surface is selected from the group consisting of a metal plate, a metal film and a metal coat.

39. The pre-existing nucleic acid of claim 19, wherein said metal is selected from the group consisting of Ag, Au, Hg, Pt, Mo and W.

40. The pre-existing nucleic acid of claim 38, wherein said metal is gold.

41. The pre-existing nucleic acid of claim 40, wherein said metal film is a thin gold film having a thickness ranging between 1 nm and 20 nm.

42. A method of covalently attaching a pre-existing nucleic acid to a metal cluster, the method comprising:
chemically modifying at least one purine or pyrimidine base of said pre-existing nucleic acid by covalently linking to said at least one purine or pyrimidine base at least one side chain terminating with at least one functional group, so as to obtain a functionallized pre-existing nucleic acid having said at least one functional group at a termini of said at least one side chain, said at least one side chain being covalently linked to said at least one purine or pyrimidine base of said pre-existing nucleic acid; and
attaching said functionallized pre-existing nucleic acid to a metal cluster, so as to covalently attach said pre-existing nucleic acid to said metal cluster via said at least one functional group.

43. The method of claim 42, wherein said pre-existing nucleic acid is RNA.

44. The method of claim 42, wherein said pre-existing nucleic acid is DNA.

45. The method of claim 42, wherein said pre-existing nucleic acid includes at least one base analog.

46. The method of claim 42, wherein said pre-existing nucleic acid is treated prior to said step of chemically modifying so as to restrict chemical modifications to predetermined purine or pyrimidine bases in said pre-existing nucleic acid.

47. The method of claim 46, wherein said pre-existing nucleic acid is a double stranded DNA and is treated by a sequence specific endonuclease prior to said step of chemically modifying so as to restrict chemical modifications to predetermined purine or pyrimidine bases in a 3′ or 5′ single stranded overhang generated by endonucleolysis.

48. The method of claim 46, wherein said pre-existing nucleic acid is a double stranded nucleic acid and is treated by a 3′ or 5′ specific exonuclease prior to said step of chemically modifying so as to restrict chemical modifications to predetermined purine or pyrimidine bases in a 5′ or 3′ single stranded overhang generated by said 3′ or 5′ specific exonucleolysis, respectively.

49. The method of claim 46, wherein said pre-existing nucleic acid is a single stranded nucleic acid and is treated by at least one complementary protecting polynucleotide prior to said step of chemically modifying so as to restrict chemical modifications to predetermined purine or pyrimidine bases in at least one region not protected by said at least one complementary protecting polynucleotide, following hybridization with said at least one complementary protecting polynucleotide.

50. The method of claim 42, wherein said chemically modifying comprises thiolating said at least one purine or pyrimidine base of said pre-existing nucleic acid.

51. The method of claim 42, wherein said functional group is a thiol group.

52. The method of claim 42, wherein said step of chemically modifying comprises thiolating said at least one purine or pyrimidine base of said pre-existing nucleic acid, so as to obtain a thiolated pre-existing nucleic acid having a thiol functional group at said termini of said side chain being covalently linked to said at least one purine or pyrimidine base of said nucleic acid.

53. The method of claim 42, wherein said side chain is saturated or unsaturated and has 2-20 carbon atoms.

54. The method of claim 53, wherein said saturated or unsaturated side chain has 2-10 carbon atoms.

55. The method of claim 53, wherein said saturated or unsaturated side chain is interrupted by at least one heteroatom selected from the group consisting of O, S and N and/or is substituted by at least one chemical group selected from the group consisting of ═O, ═NH and an alkyl group having 1-3 carbon atoms.

56. The method of claim 42, wherein said metal cluster is a gold cluster.

57. The method of claim 56, wherein said gold cluster is selected from the group consisting of a maleimido derivative of a gold cluster and colloidal gold of pre-determined size.

58. The method of claim 42, wherein said metal is selected from the group consisting of Ag, Au, Hg, Pt, Mo and W.

59. The method of claim 56, wherein said metal is gold.

60. A pre-existing nucleic acid covalently attached to a metal cluster by the method of claim 42.

61. The pre-existing nucleic acid of claim 60, interacted with a macromolecule.

62. The pre-existing nucleic acid of claim 61, wherein said macromolecule is labeled.

63. The pre-existing nucleic acid of claim 61, wherein said macromolecule is of a biological source.

64. The pre-existing nucleic acid of claim 61, wherein said macromolecule is a nucleic acid.

65. The pre-existing nucleic acid of claim 61, wherein said macromolecule is a protein.

66. The pre-existing nucleic acid of claim 60, wherein said pre-existing nucleic acid is RNA.

67. The pre-existing nucleic acid of claim 60, wherein said pre-existing nucleic acid is DNA.

68. The pre-existing nucleic acid of claim 60, wherein said pre-existing nucleic acid includes at least one base analog.

69. The pre-existing nucleic acid of claim 60, wherein said pre-existing nucleic acid is treated prior to said step of chemically modifying so as to restrict chemical modifications to predetermined purine or pyrimidine bases in said pre-existing nucleic acid.

70. The pre-existing nucleic acid of claim 69, wherein said pre-existing nucleic acid is a double stranded DNA and is treated by a sequence specific endonuclease prior to said step of chemically modifying so as to restrict chemical modifications to predetermined purine or pyrimidine bases in a 3′ or 5′ single stranded overhang generated by endonucleolysis.

71. The pre-existing nucleic acid of claim 69, wherein said pre-existing nucleic acid is a double stranded nucleic acid and is treated by a 3′ or 5′ specific exonuclease prior to said step of chemically modifying so as to restrict chemical modifications to predetermined purine or pyrimidine bases in a 5′ or 3′ single stranded overhang generated by said 3′ or 5′ specific exonucleolysis, respectively.

72. The pre-existing nucleic acid of claim 69, wherein said pre-existing nucleic acid is a single stranded nucleic acid and is treated by at least one complementary protecting polynucleotide prior to said step of chemically modifying so as to restrict chemical modifications to predetermined purine or pyrimidine bases in at least one region not protected by said at least one complementary protecting polynucleotide, following hybridization with said at least one complementary protecting polynucleotide.

73. The pre-existing nucleic acid of claim 60, wherein said chemically modifying comprises thiolating said at least one purine or pyrimidine base of said pre-existing nucleic acid.

74. The pre-existing nucleic acid of claim 60, wherein said functional group is a thiol group.

75. The pre-existing nucleic acid of claim 60, wherein said step of chemically modifying comprises thiolating said at least one purine or pyrimidine base of said pre-existing nucleic acid, so as to obtain a thiolated pre-existing nucleic acid having a thiol functional group at said termini of said side chain being covalently linked to said at least one purine or pyrimidine base of said nucleic acid.

76. The pre-existing nucleic acid of claim 60, wherein said side chain is saturated or unsaturated and has 2-20 carbon atoms.

77. The pre-existing nucleic acid of claim 76, wherein said saturated or unsaturated side chain has 2-10 carbon atoms.

78. The pre-existing nucleic acid of claim 76, wherein said saturated or unsaturated side chain is interrupted by at least one heteroatom selected from the group consisting of O, S and N and/or is substituted by at least one chemical group selected from the group consisting of ═O, ═NH and an alkyl group having 1-3 carbon atoms.

79. The pre-existing nucleic acid of claim 60, wherein said metal cluster is a gold cluster.

80. The pre-existing nucleic acid of claim 79, wherein said gold cluster is selected from the group consisting of a maleimido derivative of a gold cluster and colloidal gold of pre-determined size.

81. The pre-existing nucleic acid of claim 60, wherein said metal is selected from the group consisting of Ag, Au, Hg, Pt, Mo and W.

82. The pre-existing nucleic acid of claim 60, wherein said metal is gold.

83. A method of thiolating a pre-existing nucleic acid, the method comprising covalently attaching a side chain terminating with a thiol group to at least one unpaired purine or pyrimidine base of said pre-existing nucleic acid.

84. The method of claim 83, wherein said unpaired purine base is selected from the group consisting of an adenine base and a guanine base.

85. The method of claim 83, wherein said unpaired pyrimidine base is selected from the group consisting of a thymine base, a cytosine base and an uracil base.

86. The method of claim 83, wherein said side chain is saturated or unsaturated and has 2-20 carbon atoms.

87. The method of claim 86, wherein said saturated or unsaturated side chain has 2-10 carbon atoms.

88. The method of claim 86, wherein said saturated or unsaturated side chain is interrupted by at least one heteroatom selected from the group consisting of O, S and N and/or is substituted by at least one chemical group selected from the group consisting of ═O, ═NH and an alkyl group having 1-3 carbon atoms.

89. The method of claim 83, wherein said pre-existing nucleic acid is RNA.

90. The method of claim 83, wherein said pre-existing nucleic acid is DNA.

91. The method of claim 83, wherein said pre-existing nucleic acid includes at least one base analog.

92. The method of claim 83, wherein said pre-existing nucleic acid is treated prior to said covalently attaching, so as to obtain said at least one unpaired purine or pyrimidine base of said pre-existing nucleic acid.

93. The method of claim 92, wherein said pre-existing nucleic acid is a double stranded DNA and is treated by a sequence specific endonuclease prior to said step of covalently attaching, so as to obtain said at least one unpaired purine or pyrimidine base of said pre-existing nucleic acid in a 3′ or 5′ single stranded overhang generated by endonucleolysis.

94. The method of claim 92, wherein said pre-existing nucleic acid is a double stranded nucleic acid and is treated by a 3′ or 5′ specific exonuclease prior to said step of covalently attaching, so as to obtain said at least one unpaired purine or pyrimidine base of said pre-existing nucleic acid in a 5′ or 3′ single stranded overhang generated by said 3′ or 5′ specific exonucleolysis, respectively.

95. The method of claim 92, wherein said pre-existing nucleic acid is a single stranded nucleic acid and is treated by at least one complementary protecting polynucleotide prior to said step of covalently attaching, so as to obtain said at least one unpaired purine or pyrimidine base of said pre-existing nucleic acid in at least one region not protected by said at least one complementary protecting polynucleotide, following hybridization with said at least one complementary protecting polynucleotide.

96. A method of preparing a thin flat gold film, the method comprising evaporating a gold wire onto a chip under reduced pressure, while gradually heating said gold wire.

97. The method of claim 96, wherein said chip is a mica chip.

98. The method of claim 97, wherein said gold film has a thickness that ranges between 1 nm and 20 nm.

99. The method of claim 97, wherein said gold film has a mean roughness that ranges between 0.1 nm and 1 nm.

100. The method of claim 97, wherein said reduced pressure ranges between 10⁻⁵ torr and 10⁻⁶ torr.

101. The method of claim 97, further comprising washing said gold film with an organic solvent and drying said gold film.

102. A method of imaging a pre-existing nucleic acid, the method comprising:
chemically modifying at least one purine or pyrimidine base of said pre-existing nucleic acid by covalently linking to said at least one purine or pyrimidine base at least one side chain terminating with at least one functional group, so as to obtain a functionallized pre-existing nucleic acid having said at least one functional group at a termini of said at least one side chain, said at least one side chain being covalently linked to said at least one purine or pyrimidine base of said pre-existing nucleic acid;
covalently attaching said functionallized pre-existing nucleic acid to a metal surface, so as to obtain said pre-existing nucleic acid covalently attached to said metal surface via said at least one functional group; and
imaging said pre-existing nucleic acid covalently attached to said metal surface via said at least one functional group.

103. The method of claim 102, wherein said imaging is via atomic force microscopy.

104. The method of claim 102, wherein said imaging is via a screening method selected from the group consisting of transmission electron microscopy (TEM) scanning, dark-field scanning transmission electron microscopy (STEM), electron spectroscopic imaging (ESI), surface plasmon resonance spectroscopy (SPS) and scanning tunneling microscopy (STM).

105. The method of claim 102, wherein said pre-existing nucleic acid is RNA.

106. The method of claim 102, wherein said pre-existing nucleic acid is DNA.

107. The method of claim 102, wherein said pre-existing nucleic acid includes at least one base analog.

108. The method of claim 102, wherein said pre-existing nucleic acid is treated prior to said step of chemically modifying, so as to restrict chemical modifications to predetermined purine or pyrimidine bases in said pre-existing nucleic acid.

109. The method of claim 108, wherein said pre-existing nucleic acid is a double stranded DNA and is treated by a sequence specific endonuclease prior to said step of chemically modifying, so as to restrict chemical modifications to predetermined purine or pyrimdine bases in a 3′ or 5′ single stranded overhang generated by endonucleolysis.

110. The method of claim 108, wherein said pre-existing nucleic acid is a double stranded nucleic acid and is treated by a 3′ or 5′ specific exonuclease prior to said step of chemically modifying so as to restrict chemical modifications to predetermined purine or pyrimidine bases in a 5′ or 3′ single stranded overhang generated by said 3′ or 5′ specific exonucleolysis, respectively.

111. The method of claim 108, wherein said pre-existing nucleic acid is a single stranded nucleic acid and is treated by at least one complementary protecting polynucleotide prior to said step of chemically modifying so as to restrict chemical modifications to predetermined purine or pyrimidine bases in at least one region not protected by said at least one complementary protecting polynucleotide, following hybridization with said at least one complementary protecting polynucleotide.

112. The method of claim 102, wherein said chemically modifying comprises thiolating said at least one purine or pyrimidine base of said pre-existing nucleic acid.

113. The method of claim 102, wherein said functional group is a thiol group.

114. The method of claim 102, wherein said step of chemically modifying comprises thiolating said at least one purine or pyrimidine base of said pre-existing nucleic acid, so as to obtain a thiolated pre-existing nucleic acid having a thiol functional group at said termini of said side chain being covalently linked to said at least one purine or pyrimidine base of said nucleic acid.

115. The method of claim 102, wherein said side chain is saturated or unsaturated and has 2-20 carbon atoms.

116. The method of claim 115, wherein said saturated or unsaturated side chain has 2-10 carbon atoms.

117. The method of claim 115, wherein said saturated or unsaturated side chain is interrupted by at least one heteroatom selected from the group consisting of O, S and N and/or is substituted by at least one chemical group selected from the group consisting of ═O, ═NH and an alkyl group having 1-3 carbon atoms.

118. The method of claim 102, wherein said metal surface is selected from the group consisting of a metal plate, a metal film and a metal coat.

119. The method of claim 102, wherein said metal is selected from the group consisting of Ag, Au, Hg, Pt, Mo and W.

120. The method of claim 118, wherein said metal is gold.

121. The method of claim 120, wherein said metal film is a thin gold film having a thickness ranging between 1 nm and 20 nm.

122. The method of claim 102, wherein said metal surface is a thin flat gold film prepared by evaporating a gold wire onto a chip under reduced pressure, while gradually heating said gold wire.

123. The method of claim 122, wherein said chip is a mica chip.

124. The method of claim 122, wherein said gold film has a mean roughness that ranges between 0.1 nm and 1 nm.

125. The method of claim 122, wherein said reduced pressure ranges between 10⁻⁵ torr and 10⁻⁶ torr.

126. A method of preparing a nucleic acid chip presenting a pre-existing nucleic acid covalently attached thereto, the method comprising:
chemically modifying at least one purine or pyrimidine base of said pre-existing nucleic acid by covalently linking to said at least one purine or pyrimidine base at least one side chain terminating with at least one functional group, so as to obtain a functionallized pre-existing nucleic acid having said at least one functional group at a termini of said at least one side chain, said at least one side chain being covalently linked to said at least one purine or pyrimidine base of said pre-existing nucleic acid; and
attaching said functionallized pre-existing nucleic acid to a chip, so as to covalently attach said pre-existing nucleic acid to said chip via said at least one functional group.

127. The method of claim 126, wherein said pre-existing nucleic acid is RNA.

128. The method of claim 126, wherein said pre-existing nucleic acid is DNA.

129. The method of claim 126, wherein said pre-existing nucleic acid includes at least one base analog.

130. The method of claim 126, wherein said pre-existing nucleic acid is treated prior to said step of chemically modifying, so as to restrict chemical modifications to predetermined purine or pyrimidine bases in said pre-existing nucleic acid.

131. The method of claim 130, wherein said pre-existing nucleic acid is a double stranded DNA and is treated by a sequence specific endonuclease prior to said step of chemically modifying, so as to restrict chemical modifications to predetermined purine or pyrimidine bases in a 3′ or 5′ single stranded overhang generated by endonucleolysis.

132. The method of claim 130, wherein said pre-existing nucleic acid is a double stranded nucleic acid and is treated by a 3′ or 5′ specific exonuclease prior to said step of chemically modifying so as to restrict chemical modifications to predetermined purine or pyrimidine bases in a 5′ or 3′ single stranded overhang generated by said 3′ or 5′ specific exonucleolysis, respectively.

133. The method of claim 130, wherein said pre-existing nucleic acid is a single stranded nucleic acid and is treated by at least one complementary protecting polynucleotide prior to said step of chemically modifying so as to restrict chemical modifications to predetermined purine or pyrimidine bases in at least one region not protected by said at least one complementary protecting polynucleotide, following hybridization with said at least one complementary protecting polynucleotide.

134. The method of claim 126, wherein said chemically modifying comprises thiolating said at least one purine or pyrimidine base of said pre-existing nucleic acid.

135. The method of claim 126, wherein said functional group is a thiol group.

136. The method of claim 126, wherein said step of chemically modifying comprises thiolating said at least one purine or pyrimidine base of said pre-existing nucleic acid, so as to obtain a thiolated pre-existing nucleic acid having a thiol functional group at said termini of said side chain being covalently linked to said at least one purine or pyrimidine base of said nucleic acid.

137. The method of claim 126, wherein said side chain is saturated or unsaturated and has 2-20 carbon atoms.

138. The method of claim 137, wherein said saturated or unsaturated side chain has 2-10 carbon atoms.

139. The method of claim 137, wherein said saturated or unsaturated side chain is interrupted by at least one heteroatom selected from the group consisting of O, S and N and/or is substituted by at least one chemical group selected from the group consisting of ═O, ═NH and an alkyl group having 1-3 carbon atoms.

140. The method of claim 126, wherein said chip comprises a metal surface.

141. The method of claim 140, wherein said metal surface is selected from the group consisting of a metal plate, a metal film and a metal coat.

142. The method of claim 140, wherein said metal is selected from the group consisting of Ag, Au, Hg, Pt, Mo and W.

143. The method of claim 141, wherein said metal is gold.

144. The method of claim 143, wherein said metal film is a thin gold film having a thickness ranging between 1 nm and 20 nm.

145. A nucleic acid chip presenting a pre-existing nucleic acid covalently attached thereto, prepared by the method of claim 126.

146. The nucleic acid chip of claim 145, wherein said pre-existing nucleic acid is interacted with a macromolecule.

147. The nucleic acid chip of claim 146, wherein said macromolecule is labeled.

148. The nucleic acid chip of claim 146, wherein said macromolecule is of a biological source.

149. The nucleic acid chip of claim 146, wherein said macromolecule is a nucleic acid.

150. The nucleic acid chip of claim 146, wherein said macromolecule is a protein.

151. A method of screening a nucleic acid chip presenting a pre-existing nucleic acid covalently attached thereto and interacted with a macromolecule, the method comprising:
obtaining a nucleic acid chip presenting a pre-existing nucleic acid covalently attached thereto;
interacting said pre-existing nucleic acid with a macromolecule, so as to obtain said nucleic acid chip presenting said pre-existing nucleic acid covalently attached thereto and interacted with said macromolecule; and
screening said nucleic acid chip presenting said pre-existing nucleic acid covalently attached thereto and interacted with said macromolecule, for bound macromolecule.

152. The method of claim 151, wherein said step of obtaining said nucleic acid chip comprises:
chemically modifying at least one purine or pyrimidine base of said pre-existing nucleic acid by covalently linking to said at least one purine or pyrimidine base at least one side chain terminating with at least one functional group, so as to obtain a functionallized pre-existing nucleic acid having said at least one functional group at a termini of said at least one side chain, said at least one side chain being covalently linked to said at least one purine or pyrimidine base of said pre-existing nucleic acid; and
attaching said functionallized pre-existing nucleic acid to a metal surface, so as to covalently attach said pre-existing nucleic acid to said metal surface via said at least one functional group.

153. The method of claim 151, wherein said pre-existing nucleic acid is RNA.

154. The method of claim 151, wherein said pre-existing nucleic acid is DNA.

155. The method of claim 151, wherein said pre-existing nucleic acid includes at least one base analog.

156. The method of claim 151, wherein said chip comprises a metal surface.

157. The method of claim 156, wherein said metal surface is selected from the group consisting of a metal plate, a metal film and a metal coat.

158. The method of claim 156, wherein said metal is selected from the group consisting of Ag, Au, Hg, Pt, Mo and W.

159. The method of claim 157, wherein said metal is gold.

160. The method of claim 159, wherein said metal film is a thin gold film having a thickness ranging between 1 nm and 20 nm.

161. The method of claim 151, wherein said macromolecule is labeled.

162. The method of claim 151, wherein said macromolecule is of a biological source.

163. The method of claim 151, wherein said macromolecule is a nucleic acid.

164. The method of claim 151, wherein said macromolecule is a protein.

165. The method of claim 161, wherein said macromolecule is dye-labeled.

166. The method of claim 165, wherein said screening comprises fluorescence measurement.

167. The method of claim 161, wherein said macromolecule is labeled by a radioactive agent.

168. The method of claim 167, wherein said screening comprises autoradiographing.

169. The method of claim 161, wherein said macromolecule is labeled by a metal cluster.

170. The method of claim 169, wherein said screening is selected from the group consisting of transmission electron microscopy (TEM) scanning, dark-field scanning transmission electron microscopy (STEM) and AFM imaging.

171. The method of claim 151, wherein said screening is selected from the group consisting of AFM imaging, fluorescence measuring, autoradiographing, transmission electron microscopy (TEM) scanning, dark-field scanning transmission electron microscopy (STEM), electron spectroscopic imaging (ESI), surface plasmon resonance spectroscopy (SPS) and scanning tunneling microscopy (STM).
